(19) United States
(12) Patent Application Publication
(10) Pub. No.: US 2005/0202420 A1
(43) Pub. Date: Sep. 15, 2005

(54) Oligonucleotides or pna oligomers and a method for detecting the methylation state of genomic dna in a parallel manner

(76) Inventor: Kurt Berlin, STAHNSDORF (DE)

Correspondence Address: KRIEGSMAN & KRIEGSMAN, 665 FRANKLIN STREET, FRAMINGHAM, MA, 01702, US

(21) Appl. No.: 10/221,878

(22) PCT Filed: Mar. 15, 2001
(86) PCT No.: PCT/DE01/01089

§ 371 Date: Sep. 16, 2002

(30) Foreign Application Priority Data
Mar. 15, 2000 (DE) 100 13 847.0

(51) Int. Cl. C12Q 1/68; C07H 21/04

(52) U.S. Cl. 435/6; 536/24.3; 530/350

(57) ABSTRACT

Disclosed is a method and a set of oligonucleotides or PNA oligomers for detecting the methylation state of genomic DNA in a parallel manner. The DNA is treated with bisulphate and the thus chemically modified DNA is subsequently fragmented. In the next step, different fragments are amplified by means of synthetic primers. The amplificates are hybridized by means of oligonucleotides of a known sequence and are subsequently detected.

The present invention concerns oligonucleotides or PNA oliogmers and a method for detecting the methylation state of genomic DNA in a parallel manner.

The levels of observation that have been well studied in molecular biology according to developments in methods in recent years include the gene itself, the transcription of these genes into RNA and the translation to proteins therefrom. During the course of development of an individual, when a gene is turned on and how the activation and inhibition of certain genes in certain cells and tissues are controlled can be correlated with the extent and nature of the methylation of the genes or of the genome. Pathogenic states are also expressed by a modified methylation pattern of individual genes or of the genome.

The prior art includes methods that permit the study of methylation patterns of individual genes. More recent continuing developments of these methods also permit the analysis of minimum quantities of initial material. The present invention describes a method for the detection of the methylation state of genomic DNA samples in a parallel manner, wherein a number of different fragments of sequences that participate in gene regulation or/and transcribed and/or translated sequences that are derived from one sample are amplified simultaneously and then the sequence context of CpG dinucleotides contained in the amplified fragments is investigated.

5-Methylcytosine is the most frequent covalently modified base in the DNA of eukaryotic cells. For example, it plays a role in the regulation of transcription, genomic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

The modification of the genomic base cytosine to 5′-methylcytosine represents the most important and best-investigated epigenetic parameter up to the present time. Nevertheless, although there are presently methods for determining comprehensive genotypes of cells and individuals, there are no comparable approaches for generating and evaluating epigenotypic information on a large scale.

In principle, three different methods are known for determining the 5-methyl status of a cytosine in the sequence context.

The principle of the first method is based on the use of restriction endonucleases (REs), which are “methylation-sensitive”. REs are characterized by the fact that they introduce a cleavage in the DNA at a specific DNA sequence, for the most part between 4 and 8 bases long. The position of such cleavages can then be detected by gel electrophoresis, transfer to a membrane and hybridization. Methylation-sensitive means that specific bases must be present unmethylated within the recognition sequence, so that the cleavage can occur. The band pattern changes after a restriction cleavage and gel electrophoresis, thus depending on the methylation pattern of the DNA. Of course, the fewest methylatable CpGs are found within the recognition sequences of REs, and thus cannot be investigated according to this method.

The sensitivity of these methods is extremely low (Bird, A. P., and Southern, E. M., J. Mol. Biol. 118, 27-47). One variant combines PCR with this method; an amplification takes place by means of two primers lying on both sides of the recognition sequence after a cleavage only if the recognition sequence is present in the methylated state. The sensitivity in this case theoretically increases to a single molecule of the target sequence, but, of course, only individual positions can be investigated with high expenditure (Shemer, R. et al., PNAS 93, 6371-6376). It is again assumed that the methylatable position is found within the recognition sequence of an RE.

The second variant is based on partial chemical cleavage of total DNA, according to the prototype of a Maxam-Gilbert sequencing reaction, ligation of adaptors to the ends generated in this way, amplification with generic primers and separation by gel electrophoresis. Defined regions up to a size of less than one thousand base pairs can be investigated with this method. The method, of course, is so complicated and unreliable that it is practically no longer used (Ward, C. et al., J. Biol. Chem. 265, 3030-3033).

A relatively new method that has become the most widely used method for investigating DNA for 5-methylcytosine is based on the specific reaction of bisulfite with cytosine, which, after subsequent alkaline hydrolysis, is then converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which can now be fully utilized.

The prior art, which concerns sensitivity, is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis (Olek, A. et al., Nucl. Acids Res. 24, 5064-5066). Individual cells can be investigated by this method, which illustrates the potential of the method. Of course, up until now, only individual regions of up to approximately 3000 base pairs long have been investigated; a global investigation of cells for thousands of possible methylation events is not possible.

An overview of other known possibilities for detecting 5-methylcytosines can also be derived from the following review article: Rein, T., DePamphilis, M. L., Zorbas, H., Nucleic Acids Res. 26, 2255 (1998).

With only a few exceptions (e.g. Zeschnigk, M. et al., Eur. J. Hum. Gen. 5, 94-98; Kubota T. et al., Nat. Genet. 16, 16-17), the bisulfite technique has previously been applied only in research. However, short, specific segments of a known gene have always been amplified after a bisulfite treatment and either completely sequenced (Olek, A. and Walter, J., Nat. Genet. 17, 275-276) or individual cytosine positions are detected by a “primer extension reaction” (Gonzalgo, M. L. and Jones, P. A., Nucl. Acids Res. 25, 2529-2531) or enzyme cleavage (Xiong, Z. and Laird, P. W., Nucl. Acids Res. 25, 2532-2534). In addition, detection by hybridization has also been described (Olek et al., WO 99/28498 A1).

The methylation analysis in promoters is of particular importance for the correlation between gene activity and degree of methylation.

Common features among promoters exist not only with respect to the presence of TATA or GC boxes, but also relative to the transcription factors for which they possess binding sites and at what distance these sites are found relative to one another. The existing binding sites for a specific protein do not completely agree in their sequence, but conserved sequences of at least 4 bases are found, which can be extended still further by the insertion of “wobbles”, i.e., positions at which different bases are found each time. In addition, these binding sites are present at specific distances relative to one another.

An overview of the state of the art in oligomer array production can be taken also from a special issue of Nature Genetics which appeared in January 1999, (Nature Genetics Supplement, Volume 21, January 1999), the literature cited therein and the U.S. Pat. No. 5,994,065 on methods for the production of solid supports for target molecules such as oligonucleotides in the case of reduced nonspecific background signal.

Oligonucleotides are considered as probes which are fixed onto a surface in an oligomer array, but any modification of nucleic acids also serves for this purpose, e.g., peptide nucleic acids (PNAs), (Nielsen, P. E., Buchardt, O., Egholm, M. and Berg, R. H. 1993. Peptide nucleic acids, U.S. Pat. No. 5,539,082, Buchardt, O., Egholm M., Berg R. H. and Nielsen P. E., 1993 Peptide nucleic acids and their potential applications in biotechnology. Trends in Biotechnology, 11: 384-386), phosphorothioate oligonucleotides or methylphosphonate oligonucleotides.

Matrix-assisted laser desorption/ionization mass spectrometery (MALDI) is a new, very powerful development for the analysis of biomolecules (Karas, M. and Hillenkamp, F. 1988. Laser desorption ionization of proteins with molecular masses exceeding 10,000 daltons. Anal. Chem. 60: 2299-2301). An analyte molecule is embedded in a matrix absorbing in the UV. The matrix is vaporized in vacuum by a short laser pulse and the analyte is transported unfragmented into the gas phase. An applied voltage accelerates the ions in a field-free flight tube. Ions are accelerated to a variable extent based on their different masses. Smaller ions reach the detector earlier than larger ones and the flight time is converted into the mass of the ions.

Genomic DNA is obtained from DNA of cells, tissue or other experimental samples by standard methods. This standard methodology is found in references such as Fritsch and Maniatis, eds., Molecular Cloning, A Laboratory Manual, 1989.

Probes with multiple fluorescent labels are used for scanning an immobilized DNA array. Particularly suitable for the fluorescence label is the simple introduction of Cy3 and Cy5 dyes at the 5′OH of the respective sample. The fluorescence of the hybridized samples is detected, for example, by means of a confocal microscope. The dyes Cy3 and Cy5, in addition to many others, can be obtained commercially.

The present invention will offer a method and a set of oligonucleotides or PNA oligomers, which are suitable for the detection of the methylation state of genomic DNA in a parallel manner. Preferably, genome-wide representative CpG positions will be scanned for methylation with the use of specific, particularly suitable oligomer probes. In addition, different fragments will be amplified simultaneously from a genomic DNA sample.

The present invention describes a method and a set of oligonucleotides or PNA oligomers for the detection of the methylation state of genomic DNA in a parallel manner. The oligonucleotides or PNA oligomers originate from the following set, wherein one oligonucleotide comprises one of the following base sequences.

```
(D)₄GATGTT(D)₄; (D)₄GACGTT(D)₄; (H)₄AACATC(H)₄;
(H)₄AACGTC(H)₄;
(D)₄TTGTGA(D)₄; (D)₄TTGCGA(D)₄; (H)₄TCACAA(H)₄;
(H)₄TCGCAA(H)₄;
(D)₄TTTGAA(D)₄; (H)₄TTCAAA(H)₄; (D)₄TTCGAA(D)₄;
(H)₄TTCGAA(H)₄;
(D)₄ATTGAT(D)₄; (H)₄ATCAAT(H)₄; (D)₄ATCGAT(D)₄;
(H)₄ATCGAT(H)₄;
(D)₃TGGWTTG(D)₄; (D)₄TGGWTTG(D)₃; (D)₃CGGWTTG(D)₄;
(D)₄CGGWTTG(D)₃;
(D)₃TGGWTCG(D)₄; (D)₄TGGWTCG(D)₃; (D)₄CGGWTCG(D)₄;
(D)₄CGGWTCG(D)₃;
(H)₃CAASCCA(H)₄; (H)₄CAASCCA(H)₃; (H)₃CAASCCG(H)₄;
(H)₄CAASCCG(H)₃;
(H)₃CGASCCA(H)₄; (H)₄CGASCCA(H)₃; (H)₃CGASCGG(H)₄;
(H)₄CGASCCG(H)₃;
(D)₄AGTGTT(D)₄; (D)₄AGCGTT(D)₄; (H)₄AACACT(H)₄;
(H)₄AACGCT(H)₄;
(D)₄TATGTG(D)₄; (D)₄TACGTG(D)₄; (H)₄CACATA(H)₄;
(H)₄CACGTA(H)₄;
(D)₄TATGTA(D)₄; (H)₄TACATA(H)₄; (D)₄TACGTA(D)₄;
(H)₄TACGTA(H)₄;
(D)₄TTTGGA(D)₄; (D)₄TTCGGA(D)₄; (H)₄TCCAAA(H)₄;
(H)₄TCCGAA(H)₄;
(D)₄ATGTGT(D)₄; (H)₄ACACAT(H)₄; (D)₄ATGTGT(D)₄;
(H)₄ACGCGT(H)₄;
(D)₃GTGGTTGT(D)₃; (D)₃GCGGTTGT(D)₃;
(D)₃GCGGTCGT(D)₃;
(D)₃GTGGTCGT(D)₃;
(H)₃ACAACCAC(H)₃; (H)₃ACAACCGC(H)₃;
(H)₃ACGACCGC(H)₃;
(H)₃ACGACCAC(H)₃;
(D)₄TGTGTA(D)₄; (D)₄TGCGTA(D)₄; (H)₄TACACA(H)₄;
(H)₄TACGCA(H)₄;
(D)₄GTGTGT(D)₄; (H)₄ACACAC(H)₄; (D)₄GCGCGT(D)₄;
(H)₄ACGCGC(H)₄;
(D)₄TGTATG(D)₄; (D)₄CGTATG(D)₄; (D)₄TGTACG(D)₄;
(D)₄CGTACG(D)₄;
(H)₄CATACA(H)₄; (H)₄CATACG(H)₄; (H)₄CGTACAC(H)₄;
(H)₄CGTACG(H)₄;
(D)₄AATGTT(D)₄; (H)₄AACATT(H)₄; (D)₄AACGTT(D)₄;
(H)₄AACGTT(H)₄;
(D)₄TGATTG(D)₄; (D)₄TGATCG(D)₄; (D)₄CGATTG(D)₄;
(D)₄CGATCG(D)₄;
(H)₄CAATCA(H)₄; (H)₄CGATCA(H)₄; (H)₄CAATCG(H)₄;
(H)₄CGATCG(H)₄;
(D)₄GTTGAT(D)₄; (D)₄GTCGAT(D)₄; (H)₄ATCAAC(H)₄;
(H)₄ATCGAC(H)₄;
(D)₃TGGTATTG(D)₃; (D)₃CGGTATCG(D)₃;
(D)₃TGGTATCG(D)₃;
(D)₃CGGTATTG(D)₃;
(H)₃CAATACCA(H)₃; (H)₃CGATACCG(H)₃;
(H)₃CGATACCA(H)₃;
(H)₃CAATACCG(H)₃;
(D)₄TGTTTT(D)₄; (D)₄CGTTTT(D)₄; (H)₄AAAACA(H)₄;
(H)₄AAAACG(H)₄;
(D)₃GTGTATG(D)₄; (D)₄GTGTATG(D)₃; (D)₃GTGTACG(D)₄;
(D)₄GTGTACG(D)₃;
(H)₃CATACAC(H)₄; (H)₄CATACAC(H)₃; (H)₃CGTACAC(H)₄;
(H)₄CGTACAC(H)₃;
(D)₄GATTG(D)₅; (D)₅GATTG(D)₄; (D)₄GATCG(D)₅;
(D)₅GATCG(D)₄;
(H)₄CAATC(H)₅; (H)₅CAATC(H)₄; (H)₄CTAGC(H)₅;
(H)₅CTAGC(H)₄;
(D)₃TGTATATG(D)₃; (D)₃TGTATACG(D)₃;
(D)₃CGTATATG(D)₃;
(D)₃CGTATACG(D)₃;
(H)₃CATATACA(H)₃; (H)₃CGTATACA(H)₃;
(H)₃CATATACG(H)₃;
(H)₃CGTATACG(H)₃;
(D)₃TGAGTTTG(D)₃; (D)₃TGAGTTCG(D)₃;
(D)₃CGAGTTTG(D)₃;
(D)₃CGAGTTCG(D)₃;
(H)₃CAAACTCA(H)₃; (H)₃CGAACTCA(H)₃;
(H)₃CAAACTCG(H)₃;
(H)₃CGAACTCG(H)₃;
(D)₃TGTTAATG(D)₃; (D)₃CGTTAATG(D)₃;
(D)₃TGTTAACG(D)₃;
(D)₃CGTTAACG(D)₃;
(H)₃CATTAACA(H)₃; (H)₃CATTAACG(H)₃;
(H)₃CGTTAACA(H)₃;
(H)₃CGTTAACG(H)₃;
(D)₄TGTATG(D)₄; (D)₄TGTACG(D)₄; (D)₄CGTATG(D)₄;
(D)₄CGTACG(D)₄;
(H)₄CATACA(H)₄; (H)₄CGTACA(H)₄; (H)₄CATACG(H)₄;
(H)₄CGTACG(H)₄;
(D)₃GGTCGGTT(D)₃; (D)₃GGTTGGTT(D)₃;
(H)₃AACCGACC(H)₃;
(H)₃AACCAACC(H)₃;
(D)₄GACGT(D)₅; (D)₅GACGT(D)₄; (D)₄GATGT(D)₅;
(D)₅GATGT(D)₄;
(H)₄ACGTC(H)₅; (H)₅ACGTC(H)₄; (H)₄ACATC(H)₅;
(H)₅ACATC(H)₄;
(D)₄GGCGTT(D)₄; (D)₄GGTGTT(D)₄; (H)₄AACGCC(H)₄;
(H)₄AACACC(H)₄;
(D)₄GTCGGT(D)₄; (D)₄GTTGGT(D)₄; (H)₄ACCGAC(H)₄;
(H)₄ACCAAC(H)₄;
(D)₄TGCGG(D)₄; (D)₄TTGTGG(D)₄; (H)₄CCGCGA(H)₄;
(H)₄CCACAA(H)₄;
(D)₄TTCGGG(D)₄; (D)₄TTTGGG(D)₄; (H)₄CCCGAA(H)₄;
(H)₄CCCAAA(H)₄;
(D)₄TTCGAG(D)₄; (D)₄TTTGAG(D)₄; (H)₄CTCGAA(H)₄;
(H)₄CTCAAA(H)₄;
(D)₄CGGTCG(D)₄; (D)₄TGGTTG(D)₄; (D)₄CGGTTG(D)₄;
(D)₄TGGTCG(D)₄;
    (H)₄CGACCG(H)₄; (H)₄CAACCA(H)₄; (H)₄CAACCG(H)₄;
    (H)₄CGACCA(H)₄.
```

wherein
H is one of the bases: adenine (A), cytosine (C) or thymine (T)
D is one of the bases: adenine (A), guanine (G) or thymine (T)
W is one of the bases: adenine (A) or thymine (T)
S is one of the bases: cytosine (C) or guanine (G).

A PNA oligomer comprises one of the following sequences:

```
(D)₂GATGTT(D)₂; (D)₂GACGTT(D)₂; (H)₂AACATC(H)₂;
(H)₂AACGTC(H)₂;
(D)₂TTGTGA(D)₂; (D)₂TTGCGA(D)₂; (H)₂TCACAA(H)₂;
(H)₂TCGCAA(H)₂;
(D)₂TTTGAA(D)₂; (H)₂TTCAAA(H)₂; (D)₂TTCGAA(D)₂;
(H)₂TTCGAA(H)₂:
(D)₂ATTGAT(D)₂; (H)₂ATCAAT(H)₂; (D)₂ATCGAT(D)₂;
(H)₂ATCGAT(H)₂;
(D)₁TGGWTTG(D)₂; (D)₂TGGWTTG(D)₁; (D)₁CGGWTTG(D)₂;
(D)₂CGGWTTG(D)₁;
(D)₁TGGWTCG(D)₂; (D)₂TGGWTCG(D)₁; (D)₁CGGWTCG(D)₂;
(D)₂CGGWTCG(D)₁;
(H)₁CAASCCA(H)₂; (H)₂CAASCCA(H)₁; (H)₁CAASCCG(H)₂;
(H)₂CAASCCG(H)₁;
(H)₁CGASCCA(H)₂; (H)₂CGASCCA(H)₁; (H)₁CGASCCG(H)₂;
(H)₂CGASCCG(H)₁;
(D)₂AGTGTT(D)₂; (D)₂AGCGTT(D)₂; (H)₂AACACT(H)₂;
(H)₂AACGCT(H)₂;
(D)₂TATGTG(D)₂; (D)₂TACGTG(D)₂; (H)₂CACATA(H)₂;
(H)₂CACGTA(H)₂;
(D)₂TATGTA(D)₂; (H)₂TACATA(H)₂; (D)₂TACGTA(D)₂;
(H)₂TACGTA(H)₂;
(D)₂TTTGGA(D)₂; (D)₂TTCGGA(D)₂; (H)₂TCCAAA(H)₂;
(H)₂TCCGAA(H)₂;
(D)₂ATGTGT(D)₂; (H)₂ACACAT(H)₂; (D)₂ATGTGT(D)₂;
(H)₂ACGCGT(H)₂;
(D)₁GTGGTTGT(D)₁; (D)₁GCGGTTGT(D)₁;
(D)₁GCGGTCGT(D)₁;
(D)₁GTGGTCGT(D)₁;
(H)₁ACAACCAC(H)₁; (H)₁ACAACCGC(H)₁;
(H)₁ACGACCGC(H)₁;
(H)₁ACGACCAC(H)₁;
(D)₂TGTGTA(D)₂; (D)₂TGCGTA(D)₂; (H)₂TACACA(H)₂;
(H)₂TACGCA(H)₂;
(D)₂GTGTGT(D)₂; (H)₂ACACAC(H)₂; (D)₂GCGCGT(D)₂;
(H)₂ACGCGC(H)₂;
(D)₂TGTATG(D)₂; (D)₂CGTATG(D)₂; (D)₂TGTACG(D)₂;
(D)₂CGTACG(D)₂;
(H)₂CATACA(H)₂; (H)₂CATACG(H)₂; (H)₂CGTACA(H)₂;
(H)₂CGTACG(H)₂;
(D)₂AATGTT(D)₂; (H)₂AACATT(H)₂; (D)₂AACGTT(D)₂;
(H)₂AACGTT(H)₂;
(D)₂TGATTG(D)₂; (D)₂TGATCG(D)₂; (D)₂CGATTG(D)₂;
(D)₂CGATCG(D)₂;
(H)₂CAATCA(H)₂; (H)₂CGATCA(H)₂; (H)₂CAATCG(H)₂;
(H)₂CGATCG(H)₂;
(D)₂GTTGAT(D)₂; (D)₂GTCGAT(D)₂; (H)₂ATCAAC(H)₂;
(H)₂ATCGAC(H)₂;
(D)₁TGGTATTG(D)₁; (D)₁CGGTATCG(D)₁;
(D)₁TGGTATCG(D)₁;
(D)₁CGGTATTG(D)₁;
(H)₁CAATACCA(H)₁; (H)₁CGATACCG(H)₁;
(H)₁CGATACCA(H)₁;
(H)₁CAATACCG(H)₁;
(D)₂TGTTTT(D)₂; (D)₂CGTTTT(D)₂; (H)₂AAAACA(H)₂;
(H)₂AAAACG(H)₂;
(D)₁GTGTATG(D)₂; (D)₂GTGTATG(D)₁; (D)₁GTGTACG(D)₂;
(D)₂GTGTACG(D)₁;
(H)₃CATACAC(H)₂; (H)₂CATACAC(H)₁; (H)₁CGTACAC(H)₂;
(H)₂CGTACAC(H)₁;
(D)₂GATTG(D)₃; (D)₃GATTG(D)₂; (D)₂GATCG(D)₃;
(D)₃GATCG(D)₂;
(H)₂CAATC(H)₃; (H)₃CAATC(H)₂; (H)₂CTAGC(H)₃;
(H)₃CTAGC(H)₂;
(D)₁TGTATATG(D)₁; (D)₁TGTATACG(D)₁;
(D)₁CGTATATG(D)₁;
(D)₁CGTATACG(D)₁;
(H)₁CATATACA(H)₁; (H)₁CGTATACA(H)₁;
(H)₁CATATACG(H)₁;
(H)₁CGTATACG(H)₁;
(D)₁TGAGTTTCG(D)₁; (D)₁TGAGTTCG(D)₁;
(D)₁CGAGTTTG(D)₁;
(D)₁CGAGTTCG(D)₁;
(H)₁CAAACTCA(H)₁; (H)₁CGAACTCA(H)₁;
(H)₁CAAACTCG(H)₁;
(H)₁CGAACTCG(H)₁;
(D)₁TGTTAATG(D)₁; (D)₁CGTTAATG(D)₁;
(D)₁TGTTAACG(D)₁;
(D)₁CGTTAACG(D)₁;
(H)₁CATTAACA(H)₁; (H)₁CATTAACG(H)₁;
(H)₁CGTTAACA(H)₁;
(H)₁CGTTAACG(H)₁;
(D)₂TGTATG(D)₂; (D)₂TGTACG(D)₂; (D)₂CGTATG(D)₂;
(D)₂CGTACG(D)₂;
(H)₂CATACA(H)₂; (H)₂CGTACA(H)₂; (H)₂CATACG(H)₂;
(H)₂CGTACG(H)₂;
(D)₁GGTCGGTT(D)₁; (D)₁GGTTGGTT(D)₁;
(H)₁AACCGACC(H)₁;
(H)₁AACCAACC(H)₁;
(D)₂GACGT(D)₃; (D)₃GACGT(D)₂; (D)₂GATGT(D)₃;
(D)₃GATGT(D)₂;
(H)₂ACGTC(H)₃; (H)₃ACGTC(H)₂; (H)₂ACATC(H)₃;
(H)₃ACATC(H)₂;
(D)₂GGCGTT(D)₂; (D)₂GGTGTT(D)₂; (H)₂AACGCC(H)₂;
(H)₂AACACC(H)₂;
(D)₂GTCGGT(D)₂; (D)₂GTTGGT(D)₂; (H)₂ACCGAC(H)₂;
(H)₂ACCAAC(H)₂;
(D)₂TGCGG(D)₂; (D)₂TTGTGG(D)₂; (H)₂CCGCGA(H)₂;
(H)₂CCACAA(H)₂;
(D)₂TTCGGG(D)₂; (D)₂TTTGGG(D)₂; (H)₂CCCGAA(H)₂;
(H)₂CCCAAA(H)₂;
(D)₂TTCGAG(D)₂; (D)₂TTTGAG(D)₂; (H)₂CTCGAA(H)₂;
(H)₂CTCAAA(H)₂;
(D)₂CGGTGG(D)₂; (D)₂TGGTTG(D)₂; (D)CGGTTG(D)₂;
(D)₂TGGTCG₃(D)₂;
    (H)₂CGACCG(H)₂; (H)₂CAACCA(H)₂; (H)₂CAACCG(H)₂;
    (H)₂CGACCA(H)₂.
```

wherein
H is one of the bases: adenine (A), cytosine (C) or thymine (T)
D is one of the bases: adenine (A), guanine (G) or thymine (T)
W is one of the bases: adenine (A) or thymine (T)
S is one of the bases: cytosine (C) or guanine (G).

Several cytosine methylations in a DNA sample and preferably the upper and lower DNA strands will be analyzed simultaneously. For this purpose, the following process steps are sequentially conducted:

The genomic DNA to be analyzed is preferably obtained from the usual sources for DNA, such as, e.g., cell lines, blood, sputum, stool, urine, cerebrospinal fluid, tissue embedded in paraffin, histological slides and all possible combinations thereof.

The DNA fragments are preferably produced by digestion with one or more exonucleases or endonucleases.

In the first step of the method, a genomic DNA sample is chemically treated in such a way that cytosine bases unmethylated at the 5′ position are converted to uracil, thymine or another base dissimilar to cytosine in its hybridizing behavior.

Preferably, the above-described treatment of genomic DNA is conducted with bisultite (hydrogen sulfite, disulfite) and subsequent alkaline hydrolysis for this purpose, which leads to a conversion of unmethylated cytosine nucleobases to uracil.

In a second step of the method, more than ten different fragments of the pretreated genomic DNA are amplified simultaneously with use of synthetic oligonucleotides as primers.

These fragments preferably comprise sequences of genomic sequences that participate in gene regulation and/or transcribed and/or translated sequences as they would be present after a chemical treatment as described above.

In a preferred variant of the method, the amplification is conducted by means of the polymerase chain reaction (PCR), whereby a heat-stable DNA polymerase is used.

In another preferred variant of the method, the heat-stable DNA polymerase is selected from the following group: Taq DNA polymerase, AmpliTaq FS DNA polymerase, Deep Vent (exo.sup.-) DNA polymerase, Vent DNA polymerase, Vent (exo.sup.-) DNA polymerase and Deep Vent DNA polymerase, Thermo Sequenase, exo(-) Pseudococcus furiosus (pfu) DNA polymerase, AmpliTaq, Ultman, 9 degree Nm, Tth, Hot Tub, pyrococcus furiosus (Pfu) and Pyrococcus woesei (Pwo) DNA Polymerase.

In another preferred variant, the size of the amplified fragments in the PCR is limited to shortened chain elongation steps of less than 30 s.

In a particularly preferred variant of the method, at least one of the oligonucleotides used for the amplfication contains fewer nucleobases than would be necessary for a sequence-specific hybridization to the chemically treated genomic DNA sample.

In another particularly preferred variant of the method, at least one oligonucleotide is shorter than 18 nucleobases. In another particularly preferred variant of the method, at least one oligonucleotide is shorter than 15 nucleobases.

In another preferred variant of the method, more than 4 oligonucleotides with different sequence are used simultaneously for the amplification in one reaction vessel.

In a particularly preferred variant, more than 26 different oligonucleotides are used simultaneously for the production of a complex amplified product.

In another particularly preferred variant of the method, two oligonucleotides or two classes of oligonucleotides are used for the amplification of the described fragments, one of which or one class of which can contain the bases C, A and T, but not the base G other than in the CpG context, and the other of which or the other class of which may contain the bases G, A and T, but not the base C except in the CpG context.

In a preferred variant of the method, the products obtained after the amplification are separated by gel electrophoresis, and the fragments, which are smaller than 2000 base pairs or smaller than an arbitrary limiting value below 2000 base pairs, are separated by eliminating the other products of the amplification prior to the evaluation. These amplified products of specific size are most preferably amplified once more prior to conducting the hybridization.

In a third step of the method, the CpG dinucleotides contained in the amplified fragments are now investigated fully or partially by hybridization of the fragments, which are already provided in the amplification with a detectable label, on a set of oligonucleotides, which comprises at least two of the following sequences:

```
(D)₄GATGTT(D)₄; (D)₄GACGTT(D)₄; (H)₄AACATC(H)₄;
(H)₄AACGTC(H)₄;
(D)₄TTGTGA(D)₄; (D)₄TTGCGA(D)₄; (H)₄TCACAA(H)₄;
(H)₄TCGCAA(H)₄;
(D)₄TTTGAA(D)₄; (H)₄TTCAAA(H)₄; (D)₄TTCGAA(D)₄;
(H)₄TTCGAA(H)₄;
    (D)₄ATTGAT(D)₄; (H)₄ATCAAT(H)₄; (D)₄ATCGAT(D)₄;
    (H)₄ATCGAT(H)₄;
    (D)₃TGGWTTG(D)₄; (D)₄TGGWTTG(D)₃;
    (D)₃CGGWTTG(D)₄;
    (D)₄CGGWTTG(D)₃;
    (D)₃TGGWTCG(D)₄; (D)₄TGGWTCG(D)₃;
    (D)₃CGGWTCG(D)₄;
    (D)₄CGGWTCG(D)₃;
    (H)₃CAASCCA(H)₄; (H)₄CAASCCA(H)₃;
    (H)₃CAASCCG(H)₄;
    (H)₄CAASCCG(H)₃;
    (H)₃CGASCCA(H)₄; (H)₄CGASCCA(H)₃;
    (H)₃CGASCCG(H)₄;
    (H)₄CGASCCG(H)₃;
    (D)₄AGTGTT(D)₄; (D)₄AGCGTT(D)₄; (H)₄AACACT(H)₄;
    (H)₄AACGCT(H)₄;
    (D)₄TATGTG(D)₄; (D)₄TACGTG(D)₄; (H)₄CACATA(H)₄;
    (H)₄CACGTA(H)₄;
    (D)₄TATGTA(D)₄; (H)₄TACATA(H)₄; (D)₄TACGTA(D)₄;
    (H)₄TACGTA(H)₄;
    (D)₄TTTGGA(D)₄; (D)₄TTCGGA(D)₄; (H)₄TCCAAA(H)₄;
    (H)₄TCCGAA(H)₄;
    (D)₄ATGTGT(D)₄; (H)₄ACACAT(H)₄; (D)₄ATGTGT(D)₄;
    (H)₄ACGCGT(H)₄;
    (D)₃GTGGTTGT(D)₃; (D)₃GCGGTTGT(D)₃;
    (D)₃GCGGTCGT(D)₃;
    (D)₃GTGGTCGT(D)₃;
    (H)₃ACAACCAC(H)₃; (H)₃ACAACCGC(H)₃;
    (H)₃ACGACCGC(H)₃;
    (H)₃ACGACCAC(H)₃;
    (D)₄TGTGTA(D)₄; (D)₄TGCGTA(D)₄; (H)₄TACACA(H)₄;
    (H)₄TACGCA(H)₄;
    (D)₄GTGTGT(D)₄; (H)₄ACACAC(H)₄; (D)₄GCGCGT(D)₄;
    (H)₄ACGCGC(H)₄;
    (D)₄TGTATG(D)₄; (D)₄CGTATG(D)₄; (D)₄TGTACG(D)₄;
    (D)₄CGTACG(D)₄;
    (H)₄CATACA(H)₄; (H)₄CATACG(H)₄; (H)₄CGTACA(H)₄;
    (H)₄CGTACG(H)₄;
    (D)₄AATGTT(D)₄; (H)₄AACATT(H)₄; (D)₄AACGTT(D)₄;
    (H)₄AACGTT(H)₄;
    (D)₄TGATTG(D)₄; (D)₄TGATCG(D)₄; (D)₄CGATTG(D)₄;
    (D)₄CGATCG(D)₄;
    (H)₄CAATCA(H)₄; (H)₄CGATCA(H)₄; (H)₄CAATCG(H)₄;
    (H)₄CGATCG(H)₄;
    (D)₄GTTGAT(D)₄; (D)₄GTCGAT(D)₄; (H)₄ATCAAC(H)₄;
    (H)₄ATCGAC(H)₄;
    (D)₃TGGTATTG(D)₃; (D)₃CGGTATCG(D)₃;
    (D)₃TGGTATCG(D)₃;
    (D)₃CGGTATTG(D)₃;
    (H)₃CAATACCA(H)₃; (H)₃CGATACCG(H)₃;
    (H)₃CGATACCA(H)₃;
    (H)₃CAATACCG(H)₃;
    (D)₄TGTTTT(D)₄; (D)₄CGTTTT(D)₄; (H)₄AAAACA(H)₄;
    (H)₄AAAACG(H)₄;
    (D)₃GTGTATG(D)₄; (D)₄GTGTATG(D)₃;
    (D)₃GTGTACG(D)₄;
    (D)₄GTGTACG(D)₃;
    (H)₃CATACAC(H)₄; (H)₄CATACAC(H)₃;
    (H)₃CGTACAC(H)₄;
    (H)₄CGTACAC(H)₃;
    (D)₄GATTG(D)₅; (D)₅GATTG(D)₄; (D)₄GATCG(D)₅;
    (D)₅GATCG(D)₄;
    (H)₄CAATC(H)₅; (H)₅CAATC(H)₄; (H)₄CTAGC(H)₅;
    (H)₅CTAGC(H)₄;
    (D)₃TGTATATG(D)₃; (D)₃TGTATACG(D)₃;
    (D)₃CGTATATG(D)₃;
    (D)₃CGTATACG(D)₃;
    (H)₃CATATACA(H)₃; (H)₃CGTATACA(H)₃;
    (H)₃CATATACG(H)₃;
    (H)₃CGTATACG(H)₃;
    (D)₃TGAGTTTG(D)₃; (D)₃TGAGTTCG(D)₃;
    (D)₃CGAGTTTG(D)₃;
    (D)₃CGAGTTCG(D)₃;
    (H)₃CAAACTCA(H)₃; (H)₃CGAACTCA(H)₃;
    (H)₃CAAACTCG(H)₃;
    (H)₃CGAACTCG(H)₃;
    (D)₃TGTTAATG(D)₃; (D)₃CGTTAATG(D)₃;
    (D)₃TGTTAACG(D)₃;
    (D)₃CGTTAACG(D)₃;
    (H)₃CATTAACA(H)₃; (H)₃CATTAACG(H)₃;
    (H)₃CGTTAACA(H)₃;
    (H)₃CGTTAACG(H)₃;
    (D)₄TGTATG(D)₄; (D)₄TGTACG(D)₄; (D)₄CGTATG(D)₄;
    (D)₄CGTACG(D)₄;
    (H)₄CATACA(H)₄; (H)₄CGTACA(H)₄; (H)₄CATACG(H)₄;
    (H)₄CGTACG(H)₄;
    (D)₃GGTCGGTT(D)₃; (D)₃GGTTGGTT(D)₃;
    (H)₃AACCGACC(H)₃;
    (H)₃AACCAACC(H)₃;
    (D)₄GACGT(D)₅; (D)₅GACGT(D)₄; (D)₄GATGT(D)₅;
    (D)₃GATGT(D)₄;
    (H)₄ACGTC(H)₅; (H)₅ACGTC(H)₄; (H)₄ACATC(H)₅;
    (H)₅ACATC(H)₄;
    (D)₄GGCGTT(D)₄; (D)₄GGTGTT(D)₄; (H)₄AACGCC(H)₄;
    (H)₄AACACC(H)₄;
    (D)₄GTCGGT(D)₄; (D)₄GTTGGT(D)₄; (H)₄ACCGAC(H)₄;
    (H)₄ACCAAC(H)₄;
    (D)₄TGCGG(D)₄; (D)₄TTGTGG(D)₄; (H)₄CCGCGA(H)₄;
    (H)₄CCACAA(H)₄;
    (D)₄TTCGGG(D)₄; (D)₄TTTGGG(D)₄; (H)₄CCCGAA(H)₄;
    (H)₄CCCAAA(H)₄;
    (D)₄TTCGAG(D)₄; (D)₄TTTGAG(D)₄; (H)₄CTCGAA(H)₄;
    (H)₄CTCAAA(H)₄;
    (D)₄CGGTCG(D)₄; (D)₄TGGTTG(D)₄; (D)₄CGGTTG(D)₄;
    (D)₄TGGTCG(D)₄;
    (H)₄CGACCG(H)₄; (H)₄CAACCA(H)₄; (H)₄CAACCG(H)₄;
    (H)₄CGACCA(H)₄.
```

wherein
H is one of the bases: adenine (A), cytosine (C) or thymine (T)
D is one of the bases: adenine (A), guanine (G) or thymine (T)
W is one of the bases: adenine (A) or thymine (T)
S is one of the bases: cytosine (C) or guanine (G).

After the third method step, the CpG dinucleotides contained in the amplified fragments are investigated fully or partially by hybridization of the fragments, which are already provided with a detectable label in the amplification, on a set of PNA oligomers, which comprises at least two of the following sequences:

(D)₂GATGTT(D)₂; (D)₂GACGTT(D)₂; (H)₂AACATC(H)₂;
(H)₂AACGTC(H)₂;
(D)₂TTGTGA(D)₂; (D)₂TTGCGA(D)₂; (H)₂TCACAA(H)₂;
(H)₂TCGCAA(H)₂;
(D)₂TTTGAA(D)₂; (H)₂TTCAAA(H)₂; (D)₂TTCGAA(D)₂;
(H)₂TTCGAA(H)₂;
(D)₂ATTGAT(D)₂; (H)₂ATCAAT(H)₂; (D)₂ATCGAT(D)₂;
(H)₂ATCGAT(H)₂;
(D)₁TGGWTTG(D)₂; (D)₂TGGWTTG(D)₁; (D)₁CGGWTTG(D)₂;
(D)₂CGGWTTG(D)₁;
(D)₁TGGWTCG(D)₂; (D)₂TGGWTCG(D)₁; (D)₁CGGWTCG(D)₂;
(D)₂CGGWTCG(D)₁;
(H)₁CAASCCA(H)₂; (H)₂CAASCCA(H)₁; (H)₁CAASCCG(H)₂;
(H)₂CAASCCG(H)₁;
(H)₁CGASCCA(H)₂; (H)₂CGASCCA(H)₁; (H)₁CGASCCG(H)₂;
(H)₂CGASCCG(H)₁;
(D)₂AGTGTT(D)₂; (D)₂AGCGTT(D)₂; (H)₂AACACT(H)₂;
(H)₂AACGCT(H)₂;
(D)₂TATGTG(D)₂; (D)₂TACGTG(D)₂; (H)₂CACATA(H)₂;
(H)₂CACGTA(H)₂;
(D)₂TATGTA(D)₂; (H)₂TACATA(H)₂; (D)₂TACGTA(D)₂;
(H)₂TACGTA(H)₂;
(D)₂TTTGGA(D)₂; (D)₂TTCGGA(D)₂; (H)₂TCCAA(H)₂;
(H)₂TCCGAA(H)₂;
(D)₂ATGTGT(D)₂; (H)₂ACACAT(H)₂; (D)₂ATGTGT(D)₂;
(H)₂ACGCGT(H)₂;
(D)₁GTGGTTGT(D)₁; (D)₁GCGGTTGT(D)₁;
(D)₁GCGGTCGT(D)₁;
(D)₁GTGGTCGT(D)₁;
(H)₁ACAACCAC(H)₁; (H)₁ACAACCGC(H)₁;
(H)₁ACGACCGC(H)₁;
(H)₁ACGACCAC(H)₁;
(D)₂TGTGTA(D)₂; (D)₂TGCGTA(D)₂; (H)₂TACA(H)₂;
(H)₂TACGCA(H)₂;
(D)₂GTGTGT(D)₂; (H)₂ACACAC(H)₂; (D)₂GCGCGT(D)₂;
(H)₂ACGCGC(H)₂;
(D)₂TGTATG(D)₂; (D)₂CGTATG(D)₂; (D)₂TGTACG(D)₂;
(D)₂CGTACG(D)₂;
(H)₂CATACA(H)₂; (H)₂CATACG(H)₂; (H)₂CGTACA(H)₂;
(H)₂CGTACG(H)₂;
(D)₂AATGTT(D)₂; (H)₂AACATT(H)₂; (D)₂AACGTT(D)₂;
(H)₂AACGTT(H)₂;
(D)₂TGATTG(D)₂; (D)₂TGATCG(D)₂; (D)₂CGATTG(D)₂;
(D)₂CGATCG(D)₂;
(H)₂CAATCA(H)₂; (H)₂CGATCA(H)₂; (H)₂CAATCG(H)₂;
(H)₂CGATCG(H)₂;
(D)₂GTTGAT(D)₂; (D)₂GTCGAT(D)₂; (H)₂ATCAAC(H)₂;
(H)₂ATCGAC(H)₂;
(D)₁TGGTATTG(D)₁; (D)₁CGGTATCG(D)₁;
(D)₁TGGTATCG(D)₁;
(D)₁CGGTATTG(D)₁;
(H)₁CAATACCA(H)₁; (H)₁CGATACCG(H)₁;
(H)₁CGATACCA(H)₁;
(H)₁CAATACCG(H)₁;
(D)₂TGTTTT(D)₂; (D)₂CGTTTT(D)₂; (H)₂AAAACA(H)₂;
(H)₂AAACG(H)₂;
(D)₁GTGTATG(D)₂; (D)₂GTGTATG(D)₁; (D)₁GTGTAGG(D)₂;
(D)₂GTGTACG(D)₁;
(H)₃CATACAC(H)₂; (H)₂CATACAC(H)₁; (H)₁CGTACAC(H)₂;
(H)₂CGTACAC(H)₁;
(D)₂GATTG(D)₃; (D)₃GATTG(D)₂; (D)₂GATCG(D)₃;
(D)₃GATCG(D)₂;
(H)₂CAATC(H)₃; (H)₃CAATC(H)₂; (H)₂CTAGC(H)₃;
(H)₃CTAGC(H)₂;
(D)₁TGTATATG(D)₁; (D)₁TGTATACG(D)₁;
(D)₁CGTATATG(D)₁;
(D)₁CGTATACG(D)₁;
(H)₁CATATACA(H)₁; (H)₁CGTATACA(H)₁;
(H)₁CATATACG(H)₁;
(H)₁CGTATACG(H)₁;
(D)₁TGAGTTTG(D)₁; (D)₁TGAGTTCG(D)₁;
(D)₁CGAGTTTG(D)₁;
(D)₁CGAGTTCG(D)₁;
(H)₁CAAACTCA(H)₁; (H)₁CGAACTCA(H)₁;
(H)₁CAAACTCG(H)₁;
(H)₁CGAACTCG(H)₁;
(D)₁TGTTAATG(D)₁; (D)₁CGTTAATG(D)₁;
(D)₁TGTTAACG(D)₁;
(D)₁CGTTAACG(D)₁;
(H)₁CATTAACA(H)₁; (H)₁CATTAACG(H)₁;
(H)₁CGTTAACA(H)₁;
(H)₁CGTTAACG(H)₁;
(D)₂TGTATG(D)₂; (D)₂TGTACG(D)₂; (D)₂CGTATG(D)₂;
(D)₂CGTACG(D)₂;
(H)₂CATACA(H)₂; (H)₂CGTACA(H)₂; (H)₂CATACG(H)₂;
(H)₂CGTACG(H)₂;
(D)₁GGTCGGTT(D)₁; (D)₁GGTTGGTT(D)₁;
(H)₁AACCGACC(H)₁;
(H)₁AACCAACC(H)₁;
(D)₂GACGT(D)₃; (D)₃GACGT(D)₂; (D)₂GATGT(D)₃;
(D)₃GATGT(D)₂;
(H)₂ACGTC(H)₃; (H)₃ACGTC(H)₂; (H)₂ACATC(H)₃;
(H)₃ACATC(H)₂;
(D)₂GGCGTT(D)₂; (D)₂GGTGTT(D)₂; (H)₂AACGCC(H)₂;
(H)₂AACACC(H)₂;
(D)₂GTCGGT(D)₂; (D)₂GTTGGT(D)₂; (H)₂ACCGAC(H)₂;
(H)₂ACCAAC(H)₂;
(D)₂TGCGG(D)₂; (D)₂TTGTGG(D)₂; (H)₂CCGCGA(H)₂;
(H)₂CCACAA(H)₂;
(D)₂TTCGGG(D)₂; (D)₂TTTGGG(D)₂; (H)₂CCCGAA(H)₂;
(H)₂CCCAAA(H)₂;
(D)₂TTCGAG(D)₂; (D)₂TTTGAG(D)₂; (H)₂CTCGAA(H)₂;
(H)₂CTCAAA(H)₂;
(D)₂CGGTCG(D)₂; (D)₂TGGTTG(D)₂; (D)₂CGGTTG(D)₂;
(D)₂TGGTCG(D)₂;
(H)₂CGACCG(H)₂; (H)₂CAACCA(H)₂; (H)₂CAACCG(H)₂;
(H)₂CGACCA(H)₂.

wherein
H is one of the bases: adenine (A), cytosine (C) or thymine (T)
D is one of the bases: adenine (A), guanine (G) or thymine (T)
W is one of the bases: adenine (A) or thymine (T)
S is one of the bases: cytosine (C) or guanine (G).

In a preferred variant of the method, said fragments are investigated on an oligonucleotide array (DNA chip). The support materials are preferably selected from a group which is comprised of the following components: beads, capillaries [capillary tubes], planar support materials, membranes, wafers, silicon, glass, polystyrene, aluminum, steel, iron, copper, nickel, silver, or gold.

In a preferred variant of the method, the labels are selected from a group comprising fluorescent labels, radionuclides and detachable mass labels.

In another preferred variant of the method, the amplified fragments are immobilized on a surface and then a hybridization is conducted with a combinatory library of distinguishable oligonucleotide or PNA oligomer probes. Probes may be any nucleic acid sequences. These probes or the above-mentioned, detachable mass labels are preferably detected by means of matrix-assisted laser desorption/ionization mass spectrometry (MALDI-MS) on the basis of their unequivocal mass. Each of said probes or mass labels preferably bears a single positive or negative net charge.

Another subject of the invention is a kit, which contains at least two pairs of primers, reagents and adjuvants for the amplification and/or reagents and adjuvants for the chemical treatment and/or a combinatory probe library and/or an oligonucleotide array (DNA chip), as long as it is necessary or useful for conducting the method according to the invention.

### LIST OF ABBREVIATIONS

H is one of the bases: adenine (A), cytosine (C) or thymine (T)
D is one of the bases: adenine (A), guanine (G) or thymine (T)
W is one of the bases: adenine (A) or thymine (T)
S is one of the bases: cytosine (C) or guanine (G).

The following examples explain the invention:

### EXAMPLE 1

Conducting the Methylation Analysis of the Gene P-Cadherin

The following example relates to a fragment of the gene P-cadherin, in which a specific CG position is to be investigated for methylation.

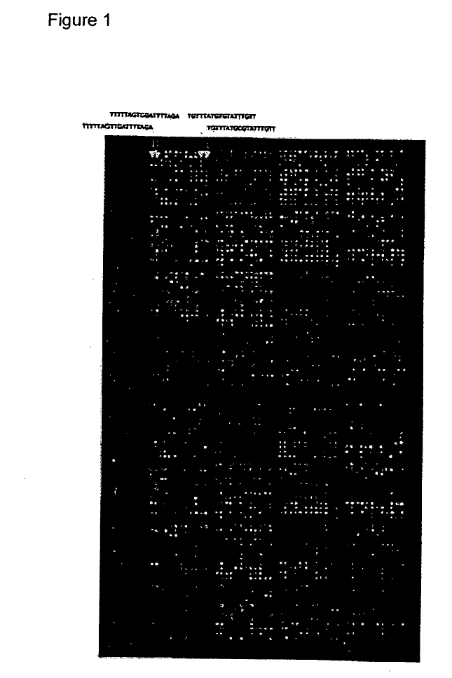

In the first step, a genomic sequence is treated with the use of bisulfite (hydrogen sulfite, disulfite) such that all of the cytosines not methylated at the 5-position of the base are modified such that a base that is different in its base-pairing behavior is formed, while the cytosines that are methylated in the 5-position remain unchanged. If bisulfite is used for the reaction, then an addition occurs at the unmethylated cytosine bases. In addition, a denaturing reagent or solvent as well as a radical trap must be present. A subsequent alkaline hydrolysis then leads to the conversion of unmethylated cytosine nucleobases to uracil. This converted DNA serves for the purpose of detecting methylated cytosines. In the second step of the method, the treated DNA sample is diluted with water or an aqueous solution. A desulfonation of the DNA is then conducted (10-30 min, 90-100° C.) at alkaline pH. In the third step of the method, the DNA sample is amplified in a polymerase chain reaction with a heat-stable DNA polymerase. In the present case, cytosines of the gene P-cadherin are investigated. For this purpose, a defined fragment of the length of 609 bp is amplified with the specific primer oligonucleotides GTTTAGAAGTTTAAGATTAG and CAAAAACTCAACCTCTATCT. This amplified product serves as the sample, which hybridizes to an oligonucleotide bound beforehand to a solid phase with the formation of a duplex structure, for example, TTTTTAGTCGATUTAGA for the methylated and TTTTTAGTTGATTTTAGA for the unmethylated state, wherein the cytosine to be detected is found at position 186 of the amplified product. The detection of the hybridization product is based on primer oligonucleotides that are fluorescently labeled with Cy3 and Cy5, which are used for the amplification (FIG. 1). Only if a methylated cytosine has been present in the bisulfite-treated DNA at this site is there a hybridization reaction of the amplified DNA with the oligonucleotide. Thus the methylation state of the respective cytosine to be investigated is decided by means of the hybridization product.

### Example 2

### Conducting the Methylation Analysis of the Gene DBCCR1

The following example refers to a fragment of the gene DBCCR1, in which a specific CG position is to be investigated for methylation.

In the first step, a genomic sequence is treated with the use of bisulfite (hydrogen sulfite, disulfite) such that all of the cytosines not methylated at the 5-position of the base are modified such that a base that is different in its base-pairing behavior is formed, while the cytosines that are methylated in the 5-position remain unchanged. If bisulfite is used for the reaction, then an addition occurs on the unmethylated cytosine bases. In addition, a denaturing reagent or solvent as well as a radical trap must be present. A subsequent alkaline hydrolysis then leads to the conversion of unmethylated cytosine nucleobases to uracil. This converted DNA serves for the purpose of detecting methylated cytosines. In the second step of the method, the treated DNA sample is diluted with water or an aqueous solution. Then a desulfonation of the DNA is conducted (10-30 min, 90-100° C.) at alkaline pH. In the third step of the method, the DNA sample is amplified in a polymerase chain reaction with a heat-stable DNA polymerase. In the present case, cytosines of the gene DBCCR1 are investigated. For this purpose, a defined fragment of the length of 531 bp is amplified with the specific primer oligonucleotides ATTTGGAGTTGAAGTATTTG and AACTATACCCAAACACCTAC. This amplified product serves as the sample, which hybridizes to an oligonucleotide bound beforehand to a solid phase with the formation of a duplex structure, here, TGTTTATGCGTATTTGTT for the methylated and TGTTTATGTGTATTTGTT for the unmethylated state, wherein the cytosine to be detected is found at position 444 of the amplified product. The detection of the hybridization product is based on primer oligonucleotides that are fluorescently labeled with Cy3 and Cy5, which were used for the amplification (FIG. 1). Only if a methylated cytosine has been present in the bisulfite-treated DNA at this site is there a hybridization reaction of the amplified DNA with the oligonucleotide. Thus the methylation state of the respective cytosine to be investigated is decided by means of the hybridization product.

### Example 3

### Conducting the Methylation Analaysis of the Gene Factor VIII

The following example refers to a fragment of the gene factor VIII, in which a specific CG position is to be investigated relative to methylation.

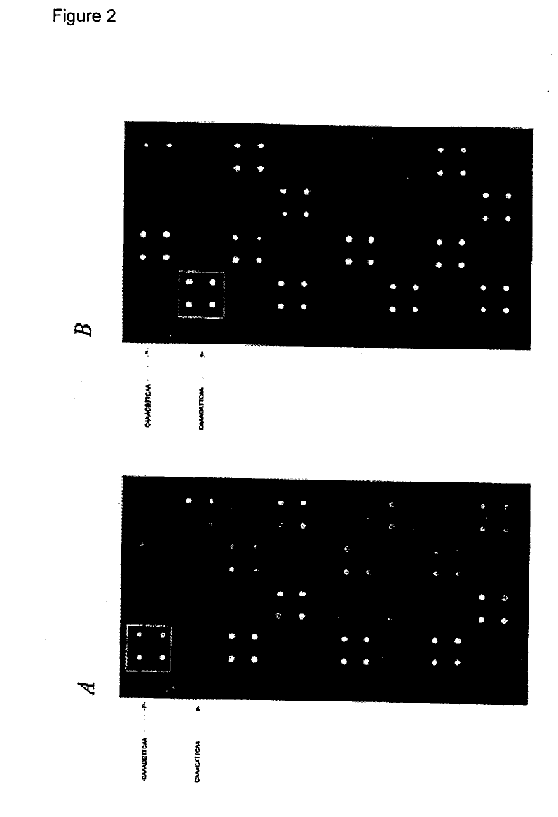

In the first step, a genomic sequence is treated with the use of bisulfite (hydrogen sulfite, disulfite) such that all of the cytosines not methylated at the 5-position of the base are modified such that a base that is different in its base-pairing behavior is formed, while the cytosines that are methylated in the 5-position remain unchanged. If bisulfite is used for the reaction, then an addition occurs at the unmethylated cytosine bases. In addition, a denaturing reagent or solvent as well as a radical trap must be present. A subsequent alkaline hydrolysis then leads to the conversion of unmethylated cytosine nucleobases to uracil. This converted DNA serves for the purpose of detecting methylated cytosines. In the second step of the method, the treated DNA sample is diluted with water or an aqueous solution. Then a desulfonation of the DNA is conducted (10-30 min, 90-100° C.) at alkaline pH. In the third step of the method, the DNA sample is amplified in a polymerase chain reaction with a heat-stable DNA polymerase. In the present case, cytosines of the gene factor VIII are investigated. For this purpose, a defined DNA fragment of the length of 561 bp is amplified with the specific primer oligonucleotides AGGGAGTTTTTTTAGGGAATAGAGGGA and TAATCCCAAAACCTCTCCACTACAACAA. This DNA amplified product serves as the sample, which hybridizes to a PNA oligonucleotide bound beforehand to a solid phase with the formation of a duplex structure, for example, CAAACGTTCAA for the methylated and CAAACATTCAA for the unmethylated state, wherein the cytosine to be detected is found at position 241 of the amplified product. The detection of the hybridization product is based on primer oligonucleotides that are fluorescently labeled with Cy3 and Cy5, which were used for the amplification (FIG. 2a, 2b). Only if a methylated cytosine has been present in the bisulfite-treated DNA at this site is there a hybridization reaction of the amplified DNA with the oligonucleotide. Thus, the methylation state of the respective cytosine to be investigated is decided by means of the hybridization product.

The figures show:

FIG. 1: Here, a high-density DNA chip is shown after hybridization. The false-color image as it is produced after scanning is represented. A color image is produced by the scanner in contrast to the black-and-white illustration shown here. The intensity of the different colors represents the degree of hybridization, whereby the degree of hybridization decreases from red (recognized as light spots in FIG. 1) to blue (recognized as dark spots in FIG. 1). The oligonucleotides are spotted in such a way that the oligonucleotides detecting the unmethylated state are found at the left (here starting at the above left each time TTTTTAGTTGATTT and TGTTTATGTGTATTTGTT) and the oligonucleotides detecting the methylated states are found at the right (also starting at the above left TTTTTAGTCGATTT or TGTTTATGCGTATTTGTT). It can be recognized that an unmethylated state can be detected each time by the oligonucleotides TTTTTAGTTGATTT and TGTTTATGTGTATTTGTT.

FIG. 2: Here, a PNA chip is shown after hybridization. The false-color image as it is produced after scanning is represented. A color image is produced by the scanner in contrast to the black-and-white illustration shown here. The intensity of the different colors represents the degree of hybridization, whereby the degree of hybridization decreases from red (recognized as light spots in FIG. 2) to blue (recognized as dark spots in FIGS. 2A and B). FIG. 2A represents the image as it occurs after scanning with the wavelength of 532 nm, which is specific for the fluorescent dye Cy3. A 4-spot PNA oligomer (CAAACGTTCAA) is found in the upper left corner, which can detect a methylated state. After hybridization with a probe, which specifically represents this state, positive signals can be recognized only at those specific positions (in the example, characterized by a white square frame). In contrast, an image is shown is shown in FIG. 2B, as it occurs after scanning with the wavelength of 635 nm, which is specific for the fluorescent dye Cy5. Here, at those positions, at which an unmethylated state can be detected with PNA oligomers (CAAACATTCAA), after hybridization with a probe representing this methylation state, positive signals can be recognized (again characterized by a white square frame for a block with four spots).

### SEQUENCE PROTOCOL

<110>Epigenomics AG
<120>Oligonucleotides or PNA oligomers and method for the detection of the methylation state of genomic DNA in a parallel manner
<130>E01-1186-WO
<140>
<141>
<160>396
<170>PatentIn Ver. 2.1
<210>1
<211>14
<212>DNA
<213>Synthetic sequence
<220>
<223>Description of the synthetic sequence: oligonucleotide
<400>1 ddddgatgtt dddd
<210>2
<211>14
<212>DNA
<213>Synthetic sequence
<220>
<223>Description of the synthetic sequence: oligonucleotide
[Key to sequence protocol on pages 23-100]
Kunstliche Sequenz=Synthetic sequence
Beschreibung der künstlichen Sequenz:Oligonukleotid=Description of the synthetic sequence: oligonucleotide

1. An oligonucleotide or PNA oliogmer for the detection of the cytosine methylation state in chemically pretreated genomic DNA, comprising one of the following base sequences:
Oligonucleotides:
(D)₄GATGTT(D)₄; (D)₄GACGTT(D)₄; (H)₄AACATC(H)₄;
(H)₄AACGTC(H)₄;
(D)₄TTGTGA(D)₄; (D)₄TTGCGA(D)₄; (H)₄TCACAA(H)₄;
(H)₄TCGCAA(H)₄;
(D)₄TTTGAA(D)₄; (H)₄TTCAAA(H)₄; (D)₄TTCGAA(D)₄;
(H)₄TTCGAA(H)₄;
(D)₄ATTGAT(D)₄; (H)₄ATCAAT(H)₄; (D)₄ATCGAT(D)₄;
(H)₄ATCGAT(H)₄;
(D)₃TGGWTTG(D)₄; (D)₄TGGWTTG(D)₃; (D)₃CGGWTTG(D)₄;
(D)₄CGGWTTG(D)₃;
(D)₃TGGWTCG(D)₄; (D)₄TGGWTCG(D)₃; (D)₃CGGWTCG(D)₄;
(D)₄CGGWTCG(D)₃;
(H)₃CAASCCA(H)₄; (H)₄CAASCCA(H)₃; (H)₃CAASCCG(H)₄;
(H)₄CAASCCG(H)₃;
(H)₃CGASCCA(H)₄; (H)₄CGASCCA(H)₃; (H)₃CGASCCG(H)₄;
(H)₄CGASCCG(H)₃;
(D)₄AGTGTT(D)₄; (D)₄AGCGTT(D)₄; (H)₄AACACT(H)₄;
(H)₄AACGCT(H)₄;
(D)₄TATGTG(D)₄; (D)₄TACGTG(D)₄; (H)₄CACATA(H)₄;
(H)₄CACGTA(H)₄;
(D)₄TATGTA(D)₄; (H)₄TACATA(H)₄; (D)₄TACGTA(D)₄;
(H)₄TACGTA(H)₄;
(D)₄TTTGGA(D)₄; (D)₄TTCGGA(D)₄; (H)₄TCCAAA(H)₄;
(H)₄TCCGAA(H)₄;
(D)₄ATGTGT(D)₄; (H)₄AGACAT(H)₄; (D)₄ATGTGT(D)₄;
(H)₄ACGCGT(H)₄;
(D)₃GTGGTTGT(D)₃; (D)₃GCGGTTGT(D)₃;
(D)₃GCGGTCGT(D)₃;
(D)₃GTGGTCGT(D)₃;
(H)₃ACAACCAC(H)₃; (H)₃ACAACCGC(H)₃;
(H)₃ACGACCGC(H)₃;
(H)₃ACGACCAC(H)₃;
(D)₄TGTGTA(D)₄; (D)₄TGCGTA(D)₄; (H)₄TACACA(H)₄;
(H)₄TACGCA(H)₄;
(D)₄GTGTGT(D)₄; (H)₄ACACAC(H)₄; (D)₄GCGCGT(D)₄;
(H)₄ACGCGC(H)₄;
(D)₄TGTATG(D)₄; (D)₄CGTATG(D)₄; (D)₄TGTACG(D)₄;
(D)₄CGTACG(D)₄;
(H)₄CATACA(H)₄; (H)₄CATACG(H)₄; (H)₄CGTACA(H)₄;
(H)₄CGTACG(H)₄;
(D)₄AATGTT(D)₄; (H)₄AACATT(H)₄; (D)₄AACGTT(D)₄;
(H)₄AACGTT(H)₄;
(D)₄TGATTG(D)₄; (D)₄TGATCG(D)₄; (D)₄CGATTG(D)₄;
(D)₄CGATCG(D)₄;
(H)₄CAATCA(H)₄; (H)₄CGATCA(H)₄; (H)₄CAATCG(H)₄;
(H)₄CGATCG(H)₄;
(D)₄GTTGAT(D)₄; (D)₄GTCGAT(D)₄; (H)₄ATCAAC(H)₄;
(H)₄ATCGAC(H)₄;
(D)₃TGGTATTG(D)₃; (D)₃CGGTATCG(D)₃;
(D)₃TGGTATCG(D)₃;
(D)₃CGGTATTG(D)₃;
(H)₃CAATACCA(H)₃; (H)₃CGATACCG(H)₃;
(H)₃CGATACCA(H)₃;
(H)₃CAATACCG(H)₃;
(D)₄TGTTTT(D)₄; (D)₄CGTTTT(D)₄; (H)₄AAAACA(H)₄;
(H)₄AAAACG(H)₄;
(D)₃GTGTATG(D)₄; (D)₄GTGTATG(D)₃; (D)₃GTGTACG(D)₄;
(D)₄GTGTACG(D)₃;
(H)₃CATACAC(H)₄; (H)₄CATACAC(H)₃; (H)₃CGTACAC(H)₄;
(H)₄CGTACAC(H)₃;
(D)₄GATTG(D)₅; (D)₅GATTG(D)₄; (D)₄GATCG(D)₅;
(D)₅GATCG(D)₄;
(H)₄CAATC(H)₅; (H)₅CAATC(H)₄; (H)₄CTAGC(H)₅;
(H)₅CTAGC(H)₄;
(D)₃TGTATATG(D)₃; (D)₃TGTATACG(D)₃;
(D)₃CGTATATG(D)₃;
(D)₃CGTATACG(D)₃;
(H)₃CATATACA(H)₃; (H)₃CGTATACA(H)₃;
(H)₃CATATACG(H)₃;
(H)₃CGTATACG(H)₃;
(D)₃TGAGTTTG(D)₃; (D)₃TGAGTTCG(D)₃;
(D)₃CGAGTTTG(D)₃;
(D)₃CGAGTTCG(D)₃;
(H)₃CAAACTCA(H)₃; (H)₃CGAACTCA(H)₃;
(H)₃CAAACTCG(H)₃;
(H)₃CGAACTCG(H)₃;
(D)₃TGTTAATG(D)₃; (D)₃CGTTAATG(D)₃;
(D)₃TGTTAACG(D)₃;
(D)₃CGTTAACG(D)₃;
(H)₃CATTAACA(H)₃; (H)₃CATTAACG(H)₃;
(H)₃CGTTAACA(H)₃;
(H)₃CGTTAACG(H)₃;
(D)₄TGTATG(D)₄; (D)₄TGTACG(D)₄; (D)₄CGTATG(D)₄;
(D)₄CGTACG(D)₄;
(H)₄CATACA(H)₄; (H)₄CGTACA(H)₄; (H)₄CATACG(H)₄;
(H)₄CGTACG(H)₄;
(D)₃GGTCGGTT(D)₃; (D)₃GGTTGGTT(D)₃;
(H)₃AACCGACG(H)₃;
(H)₃AACCAACC(H)₃;
(D)₄GACGT(D)₅; (D)₅GACGT(D)₄; (D)₄GATGT(D)₅;
(D)₅GATGT(D)₄;
(H)₄ACGTC(H)₅; (H)₅ACGTC(H)₄; (H)₄ACATC(H)₅;
(H)₅ACATC(H)₄;
(D)₄GGCGTT(D)₄; (D)₄GGTGTT(D)₄; (H)₄AACGCC(H)₄;
(H)₄AACACC(H)₄;
(D)₄GTCGGT(D)₄; (D)₄GTTGGT(D)₄; (H)₄ACCGAC(H)₄;
(H)₄ACCAAC(H)₄;
(D)₄TGCGG(D)₄; (D)₄TTGTGG(D)₄; (H)₄CCGCGA(H)₄;
(H)₄CCACAA(H)₄;
(D)₄TTCGGG(D)₄; (D)₄TTTGGG(D)₄; (H)₄CCCGAA(H)₄;
(H)₄CCCAAA(H)₄;
(D)₄TTCGAG(D)₄; (D)₄TTTGAG(D)₄; (H)₄CTCGAA(H)₄;
(H)₄CTCAAA(H)₄;
(D)₄CGGTCG(D)₄; (D)₄TGGTTG(D)₄; (D)₄CGGTTG(D)₄;
(D)₄TGGTCG(D)₄;
(H)₄CGACCG(H)₄; (H)₄CAACCA(H)₄; (H)₄CAACCG(H)₄;
(H)₄CGACCA(H)₄.
wherein
H is one of the bases: adenine (A), cytosine (C) or thymine (T)
D is one of the bases: adenine (A), guanine (G) or thymine (T)
W is one of the bases: adenine (A) or thymine (T)
S is one of the bases: cytosine (C) or guanine (G).
PNA oligomers:
(D)₂GATGTT(D)₂; (D)₂GACGTT(D)₂; (H)₂AACATC(H)₂;
(H)₂AACGTC(H)₂;
(D)₂TTGTGA(D)₂; (D)₂TTGCGA(D)₂; (H)₂TCACAA(H)₂;
(H)₂TCGCAA(H)₂;
(D)₂TTTGAA(D)₂; (H)₂TTCAAA(H)₂; (D)₂TTCGAA(D)₂;
(H)₂TCGAA(H)₂;
(D)₂ATTGAT(D)₂; (H)₂ATCAAT(H)₂; (D)₂ATCGAT(D)₂;
(H)₂ATCGAT(H)₂;
(D)₁TGGWTTG(D)₂; (D)₂TGGWTTG(D)₁; (D)₁CGGWTTG(D)₂;
(D)₂CGGWTTG(D)₁;
(D)₁TGGWTCG(D)₂; (D)₂TGGWTCG(D)₁; (D)₁CGGWTCG(D)₂;
(D)₂CGGWTCG(D)₁;
(H)₁CAASCCA(H)₂; (H)₂CAASCCA(H)₁; (H)₁CAASCCG(H)₂;
(H)₂CAASCCG(H)₁;
(H)₁CGASCCA(H)₂; (H)₂CGASCCA(H)₁; (H)₁CGASCCG(H)₂;
(H)₂CGASCGG(H)₁;
(D)₂AGTGTT(D)₂; (D)₂AGCGTT(D)₂; (H)₂AACACT(H)₂;
(H)₂ACGCT(H)₂;
(D)₂TATGTG(D)₂; (D)₂TACGTG(D)₂; (H)₂CACATA(H)₂;
(H)₂CACGTA(H)₂;
(D)₂TATGTA(D)₂; (H)₂TACATA(H)₂; (D)₂TACGTA(D)₂;
(H)₂TACGTA(H)₂;
(D)₂TTTGGA(D)₂; (D)₂TTCGGA(D)₂; (H)₂TCCAAA(H)₂;
(H)₂TCCGAA(H)₂;
(D)₂ATGTGT(D)₂; (H)₂ACACAT(H)₂; (D)₂ATGTGT(D)₂;
(H)₂ACGCGT(H)₂;
(D)₁GTGGTTGT(D)₁; (D)₁GCGGTTGT(D)₁;
(D)₁GCGGTCGT(D)₁;
(D)₁GTGGTCGT(D)₁;
(H)₁ACAACCAC(H)₁; (H)₁ACAACCGC(H)₁;
(H)₁ACGACCGC(H)₁;
(H)₁ACGAGCAC(H)₁;
(D)₂TGTGTA(D)₂; (D)₂TGCGTA(D)₂; (H)₂TACACA(H)₂;
(H)₂TACGCA(H)₂;
(D)₂GTGTGT(D)₂; (H)₂ACACAC(H)₂; (D)₂GCGCGT(D)₂;
(H)₂ACGCGC(H)₂;
(D)₁GTGGTTGT(D)₁; (D)₁GCGGTTGT(D)₁;
(D)₁GCGGTCGT(D)₁;
(D)₁GTGGTCGT(D)₁;
(H)₁ACAACCAC(H)₁; (H)₁ACAACCGC(H)₁;
(H)₁ACGACGGC(H)₁;
(H)₁ACGACCAC(H)₁;
(D)₂TGTGTA(D)₂; (D)₂TGCGTA(D)₂; (H)₂TACACA(H)₂;
(H)₂TACGCA(H)₂;
(D)₂GTGTGT(D)₂; (H)₂ACACAC(H)₂; (D)₂GCGCGT(D)₂;
(H)₂ACGCGC(H)₂;
(D)₂TGTATG(D)₂; (D)₂CGTATG(D)₂; (D)₂TGTACG(D)₂;
(D)₂CGTACG(D)₂;
(H)₂CATACA(H)₂; (H)₂CATACG(H)₂; (H)₂CGTACA(H)₂;
(H)₂CGTACG(H)₂;
(D)₂AATGTT(D)₂; (H)₂AACATT(H)₂; (D)₂AACGTT(D)₂;
(H)₂AACGTT(H)₂;
(D)₂TGATTG(D)₂; (D)₂TGATCG(D)₂; (D)₂CGATTG(D)₂;
(D)₂CGATCG(D)₂;
(H)₂CAATCA(H)₂; (H)₂CGATCA(H)₂; (H)₂CAATCG(H)₂;
(H)₂CGATCG(H)₂;
(D)₂GTTGAT(D)₂; (D)₂GTCGAT(D)₂; (H)₂ATCAAC(H)₂;
(H)₂ATCGAC(H)₂;
(D)₁TGGTATTG(D)₁; (D)₁CGGTATCG(D)₁;
(D)₁TGGTATCG(D)₁;
(D)₁CGGTATTG(D)₁;
(H)₁CAATACCA(H)₁; (H)₁CGATACGG(H)₁;
(H)₁CGATACCA(H)₁;
(H)₁CAATACCG(H)₁;
(D)₂TGTTTT(D)₂; (D)₂CGTTTT(D)₂; (H)₂AAAACA(H)₂;
(H)₂AAAACG(H)₂;
(D)₁GTGTATG(D)₂; (D)₂GTGTATG(D)₁; (D)₁GTGTACG(D)₂;
(D)₂GTGTACG(D)₁;
(H)₃CATACAC(H)₂; (H)₂CATACAC(H)₁; (H)₁CGTACAC(H)₂;
(H)₂CGTACAC(H)₁;
(D)₂GATTG(D)₃; (D)₃GATTG(D)₂; (D)₂GATCG(D)₃;
(D)₃GATCG(D)₂;
(H)₂CAATC(H)₃; (H)₃CAATC(H)₂; (H)₂CTAGC(H)₃;
(H)₃CTAGC(H)₂;
(D)₁TGTATATG(D)₁; (D)₁TGTATACG(D)₁;
(D)₁CGTATATG(D)₁;
(D)₁CGTATACG(D)₁;
(H)₁CATATACA(H)₁; (H)₁CGTATACA(H)₁;
(H)₁CATATACG(H)₁;
(H)₁CGTATACG(H)₁;
(D)₁TGAGTTTG(D)₁; (D)₁TGAGTTCG(D)₁;
(D)₁CGAGTTTG(D)₁;
(D)₁CGAGTTCG(D)₁;
(H)₁CAAACTCA(H)₁; (H)₁CGAACTCA(H)₁;
(H)₁CAAACTCG(H)₁;
(H)₁CGAACTCG(H)₁;
(D)₁TGTTAATG(D)₁; (D)₁CGTTAATG(D)₁;
(D)₁TGTTAAGG(D)₁;
(D)₁CGTTAACG(D)₁;
(H)₁CATTAACA(H)₁; (H)₁CATTAACG(H)₁;
(H)₁CGTTAACA(H)₁;
(H)₁GGTTAACG(H)₁;
(D)₂TGTATG(D)₂; (D)₂TGTACG(D)₂; (D)₂CGTATG(D)₂;
(D)₂CGTACG(D)₂;
(H)₂CATACA(H)₂; (H)₂CGTACA(H)₂; (H)₂CATACG(H)₂;
(H)₂CGTACG(H)₂;
(D)₁GGTCGGTT(D)₁; (D)₁GGTTGGTT(D)₁;
(H)₁AACCGACC(H)₁;
(H)₁AACCAACC(H)₁;
(D)₂GACGT(D)₃; (D)₃GACGT(D)₂; (D)₂GATGT(D)₃;
(D)₃GATGT(D)₂;
(H)₂ACGTC(H)₃; (H)₃ACGTC(H)₂; (H)₂ACATC(H)₃;
(H)₃ACATC(H)₂;
(D)₂GGCGTT(D)₂; (D)₂GGTGTT(D)₂; (H)₂AACGCC(H)₂;
(H)₂AACACC(H)₂;
(D)₂GTCGGT(D)₂; (D)₂GTTGGT(D)₂; (H)₂ACCGAC(H)₂;
(H)₂ACCAAC(H)₂;
(D)₂TGCGG(D)₂; (D)₂TTGTGG(D)₂; (H)₂CCGCGA(H)₂;
(H)₂CCACAA(H)₂;
(D)₂TTCGGG(D)₂; (D)₂TTTGGG(D)₂; (H)₂CCCGAA(H)₂;
(H)₂CCCAAA(H)₂;
(D)₂TTCGAG(D)₂; (D)₂TTTGAG(D)₂; (H)₂CTCGAA(H)₂;
(H)₂CTCAAA(H)₂;
(D)₂CGGTCG(D)₂; (D)₂TGGTTG(D)₂; (D)₂CGGTTG(D)₂;
(D)₂TGGTCG(D)₂;
(H)₂CGACCG(H)₂; (H)₂CAACCA(H)₂; (H)₂CAACCG(H)₂;
(H)₂CGACCA(H)₂.
wherein
H is one of the bases: adenine (A), cytosine (C) or thymine (T)
D is one of the bases: adenine (A), guanine (G) or thymine (T)
W is one of the bases: adenine (A) or thymine (T)
S is one of the bases: cytosine (C) or guanine (G).

2. Use of a set of oligonucleotides comprising at least two of the sequences of list 1, according to claim 1, for the detection of cytosine methylations in DNA samples.

3. A method for the detection of the methylation state of genomic DNA in a parallel manner, characterized in that the following steps are conducted:
a) unmethylated cytosine bases at the 5′-position in a genomic DNA sample are converted by chemical treatment to uracil, thymidine or another base that is dissimilar to cytosine in its hybridization behavior;
b) more than ten different fragments, each of which is less than 2000 base pairs long, are amplified from this chemically treated genomic DNA with use of synthetic oligonucleotides as primers;
c) the amplified products are hybridized to a set of oligonucleotides or PNA oligomers, which comprises at least two of the following sequences:
c1) oligonucleotides
(D)₄GATGTT(D)₄; (D)₄GACGTT(D)₄; (H)₄AACATC(H)₄;
(H)₄AACGTC(H)₄;
(D)₄TTGTGA(D)₄; (D)₄TTGCGA(D)₄; (H)₄TCACAA(H)₄;
(H)₄TCGCAA(H)₄;
(D)₄TTTGAA(D)₄; (H)₄TTGAAA(H)₄; (D)₄TTCGAA(D)₄;
(H)₄TTCGAA(H)₄;
(D)₄ATTGAT(D)₄; (H)₄ATCAAT(H)₄; (D)₄ATCGAT(D)₄;
(H)₄ATCGAT(H)₄;
(D)₃TGGWTTG(D)₄; (D)₄TGGWTTG(D)₃; (D)₃CGGWTTG(D)₄;
(D)₄CGGWTTG(D)₃;
(D)₃TGGWTCG(D)₄; (D)₄TGGWTCG(D)₃; (D)₃CGGWTCG(D)₄;
(D)₄GGGWTCG(D)₃;
(H)₃CAASCCA(H)₄; (H)₄CAASCCA(H)₃; (H)₃CAASCCG(H)₄;
(H)₄CAASCCG(H)₃;
(H)₃CGASCCA(H)₄; (H)₄CGASCCA(H)₃; (H)₃CGASCCG(H)₄;
(H)₄CGASCCG(H)₃;
(D)₄AGTGTT(O)₄; (D)₄AGCGTT(D)₄; (H)₄AACACT(H)₄;
(H)₄AACGCT(H)₄;
(D)₄TATGTG(D)₄; (D)₄TACGTG(D)₄; (H)₄CACATA(H)₄;
(H)₄CACGTA(H)₄;
(D)₄TATGTA(D)₄; (H)₄TACATA(H)₄; (D)₄TACGTA(D)₄;
(H)₄TACGTA(H)₄;
(D)₄TTTGGA(D)₄; (D)₄TTCGGA(D)₄; (H)₄TCCAAA(H)₄;
(H)₄TCCGAA(H)₄;
(D)₄ATGTGT(D)₄; (H)₄ACACAT(H)₄; (D)₄ATGTGT(D)₄;
(H)₄ACGCGT(H)₄;
(D)₃GTGGTTGT(D)₃; (D)₃GCGGTTGT(D)₃;
(D)₃GCGGTCGT(D)₃;
(D)₃GTGGTCGT(D)₃;
(H)₃ACAACCAC(H)₃; (H)₃ACAACCGC(H)₃;
(H)₃ACGACCGC(H)₃;
(H)₃ACGACCAC(H)₃;
(O)₄TGTGTA(D)₄; (D)₄TGCGTA(D)₄; (H)₄TACACA(H)₄;
(H)₄TACGCA(H)₄;
(D)₄GTGTGT(D)₄; (H)₄ACACAC(H)₄; (D)₄GCGCGT(D)₄;
(H)₄ACGCGC(H)₄;
(D)₄TGTATG(D)₄; (D)₄CGTATG(D)₄; (D)₄TGTACG(D)₄;
(D)₄CGTACG(D)₄;
(H)₄CATACA(H)₄; (H)₄CATACG(H)₄; (H)₄CGTACA(H)₄;
(H)₄CGTACG(H)₄;
(D)₄AATGTT(D)₄; (H)₄AACATT(H)₄; (D)₄AACGTT(D)₄;
(H)₄AACGTT(H)₄;
(D)₄TGATTG(D)₄; (D)₄TGATCG(D)₄; (D)₄CGATTG(D)₄;
(D)₄CGATCG(D)₄;
(H)₄CAATCA(H)₄; (H)₄CGATCA(H)₄; (H)₄CAATCG(H)₄;
(H)₄CGATCG(H)₄;
(D)₄GTTGAT(D)₄; (D)₄GTCGAT(D)₄; (H)₄ATCAAC(H)₄;
(H)₄ATCGAC(H)₄;
(D)₃TGGTATTG(D)₃; (D)₃CGGTATCG(D)₃;
(D)₃TGGTATCG(D)₃;
(D)₃CGGTATTG(D)₃;
(H)₃CAATACCA(H)₃; (H)₃CGATACCG(H)₃;
(H)₃CGATACCA(H)₃;
(H)₃CAATACCG(H)₃;
(D)₄TGTTTT(D)₄; (D)₄CGTTTT(D)₄; (H)₄AAAACA(H)₄;
(H)₄AAAACG(H)₄;
(D)₃GTGTATG(D)₄; (D)₄GTGTATG(D)₃; (D)₃GTGTACG(D)₄;
(D)₄GTGTACG(D)₃;
(H)₃CATACAC(H)₄; (H)₄CATACAC(H)₃; (H)₃CGTACAC(H)₄;
(H)₄CGTACAC(H)₃;
(D)₄GATTG(D)₅; (D)₅GATTG(D)₄; (D)₄GATCG(D)₅;
(D)₅GATCG(D)₄;
(H)₄CAATC(H)₅; (H)₅CAATC(H)₄; (H)₄CTAGC(H)₅;
(H)₅CTAGC(H)₄;
(D)₃TGTATATG(D)₃; (D)₃TGTATACG(D)₃;
(D)₃CGTATATG(D)₃;
(D)₃CGTATACG(D)₃;
(H)₃CATATACA(H)₃; (H)₃CGTATACA(H)₃;
(H)₃CATATACG(H)₃;
(H)₃CGTATACG(H)₃;
(D)₃TGAGTTTG(D)₃; (D)₃TGAGTTCG(D)₃;
(D)₃CGAGTTTG(D)₃;
(D)₃CGAGTTCG(D)₃;
(H)₃CAAACTCA(H)₃; (H)₃CGAACTCA(H)₃;
(H)₃CAAACTCG(H)₃;
(H)₃CGAACTCG(H)₃;
(D)₃TGTTAATG(D)₃; (D)₃CGTTAATG(D)₃;
(D)₃TGTTAACG(D)₃;
(D)₃CGTTAACG(D)₃;
(H)₃CATTAACA(H)₃; (H)₃CATTAACG(H)₃;
(H)₃CGTTAACA(H)₃;
(H)₃CGTTAACG(H)₃;
(D)₄TGTATG(D)₄; (D)₄TGTACG(D)₄; (D)₄CGTATG(D)₄;
(D)₄CGTACG(D)₄;
(H)₄CATACA(H)₄; (H)₄CGTACA(H)₄; (H)₄CATACG(H)₄;
(H)₄CGTACG(H)₄;
(D)₃GGTCGGTT(D)₃; (D)₃GGTTGGTT(D)₃;
(H)₃AACCGACC(H)₃;
(H)₃AACCAACC(H)₃;
(D)₄GACGT(D)₅; (D)₅GACGT(D)₄; (D)₄GATGT(D)₅;
(D)₅GATGT(D)₄;
(H)₄ACGTC(H)₅; (H)₅ACGTC(H)₄; (H)₄ACATC(H)₅;
(H)₅ACATC(H)₄;
(D)₄GGCGTT(D)₄; (D)₄GGTGTT(D)₄; (H)₄AACGCC(H)₄;
(H)₄AACACC(H)₄;
(D)₄GTCGGT(D)₄; (D)₄GTTGGT(D)₄; (H)₄ACCGAC(H)₄;
(H)₄ACCAAC(H)₄;
(D)₄TGCGG(D)₄; (D)₄TTGTGG(D)₄; (H)₄CCGCGA(H)₄;
(H)₄CCACAA(H)₄;
(D)₄TTCGGG(D)₄; (D)₄TTTGGG(D)₄; (H)₄CCCGAA(H)₄;
(H)₄CCGAAA(H)₄;
(D)₄TTCGAG(D)₄; (D)₄TTTGAG(D)₄; (H)₄CTCGAA(H)₄;
(H)₄CTCAAA(H)₄;
(D)₄CGGTCG(D)₄; (D)₄TGGTTG(D)₄; (D)₄CGGTTG(D)₄;
(D)₄TGGTCG(D)₄;
(H)₄CGACCG(H)₄; (H)₄CAACCA(H)₄; (H)₄CAACCG(H)₄;
(H)₄CGACCA(H)₄.
wherein
H is one of the bases: adenine (A), cytosine (C) or thymine (T)
D is one of the bases: adenine (A), guanine (G) or thymine (T)
W is one of the bases: adenine (A) or thymine (T)
S is one of the bases: cytosine (C) or guanine (G),
c2) PNA oligomers:
```
    (D)₂GATGTT(D)₂; (D)₂GACGTT(D)₂; (H)₂AACATC(H)₂;
    (H)₂AACGTC(H)₂;
(D)₂TTGTGA(D)₂; (D)₂TTGCGA(D)₂; (H)₂TCACAA(H)₂;
(H)₂TCGCAA(H)₂;
(D)₂TTTGAA(D)₂; (H)₂TTCAAA(H)₂; (D)₂TTCGAA(D)₂;
(H)₂TTCGAA(H)₂;
(D)₂ATTGAT(D)₂; (H)₂ATCAAT(H)₂; (D)₂ATCGAT(D)₂;
(H)₂ATCGAT(H)₂;
(D)₁TGGWTTG(D)₂; (D)₂TGGWTTG(D)₁; (D)₁CGGWTTG(D)₂;
(D)₂CGGWTTG(D)₁;
(D)₁TGGWTCG(D)₂; (D)₂TGGWTCG(D)₁; (D)₁CGGWTCG(D)₂;
(D)₂CGGWTCG(D)₁;
(H)₁CAASCCA(H)₂; (H)₂CAASCCA(H)₁; (H)₁CAASCCG(H)₂;
(H)₂CAASCCG(H)₁;
(H)₁CGASCCA(H)₂; (H)₂CGASCCA(H)₁; (H)₁CGASCCG(H)₂;
(H)₂CGASCCG(H)₁;
D)₂AGTGTT(D)₂; (D)₂AGCGTT(D)₂; (H)₂AACACT(H)₂;
(H)₂AACGCT(H)₂;
(D)₂TATGTG(D)₂; (D)₂TACGTG(D)₂; (H)₂CACATA(H)₂;
(H)₂CACGTA(H)₂;
(D)₂TATGTA(D)₂; (H)₂TACATA(H)₂; (D)₂TACGTA(D)₂;
(H)₂TACGTA(H)₂;
(D)₂TTTGGA(D)₂; (D)₂TTCGGA(D)₂; (H)₂TCCAAA(H)₂;
(H)₂TCCGAA(H)₂;
(D)₂ATGTGT(D)₂; (H)₂ACACAT(H)₂; (D)₂ATGTGT(D)₂;
(H)₂ACGCGT(H)₂;
(D)₁GTGGTTGT(D)₁; (D)₁GCGGTTGT(D)₁;
(D)₁GCGGTCGT(D)₁;
(D)₁GTGGTCGT(D)₁;
(H)₁ACAACCAC(H)₁; (H)₁ACAACCGC(H)₁;
(H)₁ACGACCGC(H)₁;
(H)₁ACGACCAC(H)₁;
(D)₂TGTGTA(D)₂; (D)₂TGCGTA(D)₂; (H)₂TACACA(H)₂;
(H)₂TACGCA(H)₂;
(D)₂GTGTGT(D)₂; (H)₂ACACAC(H)₂; (D)₂GCGCGT(D)₂;
(H)₂ACGCGC(H)₂;
(D)₂TGTATG(D)₂; (D)₂CGTATG(D)₂; (D)₂TGTACG(D)₂;
(D)₂CGTACG(D)₂;
(H)₂CATACA(H)₂; (H)₂CATACG(H)₂; (H)₂CGTACA(H)₂;
(H)₂CGTACG(H)₂;
(D)₂AATGTT(D)₂; (H)₂AACATT(H)₂; (D)₂AACGTT(D)₂;
(H)₂AACGTT(H)₂;
(D)₂TGATTG(D)₂; (D)₂TGATCG(D)₂; (D)₂CGATTG(D)₂;
(D)₂CGATCG(D)₂;
(H)₂CAATCA(H)₂; (H)₂CGATCA(H)₂; (H)₂CAATCG(H)₂;
(H)₂CGATCG(H)₂;
(D)₂GTTGAT(D)₂; (D)₂GTCGAT(D)₂; (H)₂ATCAAC(H)₂;
(H)₂ATCGAC(H)₂;
(D)₁TGGTATTG(D)₁; (D)₁CGGTATCG(D)₁;
(D)₁TGGTATCG(D)₁;
(D)₁CGGTATTG(D)₁;
(H)₁CAATACCA(H)₁; (H)₁CGATACCG(H)₁;
(H)₁CGATACCA(H)₁;
(H)₁CAATACCG(H)₁;
(D)₂TGTTTT(D)₂; (D)₂CGTTTT(D)₂; (H)₂AAAACA(H)₂;
(H)₂AAAACG(H)₂;
(D)₁GTGTATG(D)₂; (D)₂GTGTATG(D)₁; (D)₁GTGTACG(D)₂;
(D)₂GTGTACG(D)₁;
(H)₃CATACAC(H)₂; (H)₂CATACAC(H)₁; (H)₁CGTACAC(H)₂;
(H)₂CGTACAC(H)₁;
(D)₂GATTG(D)₃; (D)₃GATTG(D)₂; (D)₂GATCG(D)₃;
(D)₃GATCG(D)₂;
(H)₂CAATC(H)₃; (H)₃CAATC(H)₂; (H)₂CTAGC(H)₃;
(H)₃CTAGC(H)₂;
(D)₁TGTATATG(D)₁; (D)₁TGTATACG(D)₁;
(D)₁CGTATATG(D)₁;
(D)₁CGTATACG(D)₁;
(H)₁CATATACA(H)₁; (H)₁CGTATACA(H)₁;
(H)₁CATATACG(H)₁;
(H)₁CGTATACG(H)₁;
(D)₁TGAGTTTG(D)₁; (D)₁TGAGTTCG(D)₁;
(D)₁CGAGTTTG(D)₁;
(D)₁CGAGTTCG(D)₁;
(H)₁CAAACTCA(H)₁; (H)₁CGAACTCA(H)₁;
(H)₁CAAACTCG(H)₁;
(H)₁CGAACTCG(H)₁;
(D)₁TGTTAATG(D)₁; (D)₁CGTTAATG(D)₁;
(D)₁TGTTAACG(D)₁;
(D)₁CGTTAACG(D)₁;
(H)₁CATTAACA(H)₁; (H)₁CATTAACG(H)₁;
(H)₁CGTTAACA(H)₁;
(H)₁CGTTAACG(H)₁;
(D)₂TGTATG(D)₂; (D)₂TGTACG(D)₂; (D)₂CGTATG(D)₂;
(D)₂CGTACG(D)₂;
(H)₂CATACA(H)₂; (H)₂CGTACA(H)₂; (H)₂CATACG(H)₂;
(H)₂CGTACG(H)₂;
(D)₁GGTCGGTT(D)₁; (D)₁GGTTGGTT(D)₁;
(H)₁AACCGACC(H)₁;
(H)₁AACCAACC(H)₁;
(D)₂GACGT(D)₃; (D)₃GACGT(D)₂; (D)₂GATGT(D)₃;
(D)₃GATGT(D)₂;
(H)₂ACGTC(H)₃; (H)₃ACGTC(H)₂; (H)₂ACATC(H)₃;
(H)₃ACATC(H)₂;
(D)₂GGCGTT(D)₂; (D)₂GGTGTT(D)₂; (H)₂AACGCC(H)₂;
(H)₂AACACC(H)₂;
(D)₂GTCGGT(D)₂; (D)₂GTTGGT(D)₂; (H)₂ACCGAC(H)₂;
(H)₂ACCAAC(H)₂;
(D)₂TGCGG(D)₂; (D)₂TTGTGG(D)₂; (H)₂CCGCGA(H)₂;
(H)₂CCACAA(H)₂;
(D)₂TTCGGG(D)₂; (D)₂TTTGGG(D)₂; (H)₂CCCGAA(H)₂;
(H)₂CCCAAA(H)₂;
(D)₂TTCGAG(D)₂; (D)₂TTTGAG(D)₂; (H)₂CTCGAA(H)₂;
(H)₂CTCAAA(H)₂;
(D)₂CGGTCG(D)₂; (D)₂TGGTTG(D)₂; (D)₂CGGTTG(D)₂;
(D)₂TGGTCG(D)₂;
(H)₂CGACCG(H)₂; (H)₂CAACCA(H)₂; (H)₂CAACCG(H)₂;
(H)₂CGACCA(H)₂.
```
wherein
H is one of the bases: adenine (A), cytosine (C) or thymine (T)
D is one of the bases: adenine (A), guanine (G) or thymine (T)
W is one of the bases: adenine (A) or thymine (T)
S is one of the bases: cytosine (C) or guanine (G),
d) the hybridized amplified products are detected.

4. The method according to claim 3, further characterized in that the chemical treatment is conducted by means of a solution of a bisulfite, hydrogen sulfite or disulfite.

5. The method according to claim 3 or 4, further characterized in that the amplification is conducted by means of the polymerase chain reaction (PCR).

6. The method according to claim 3, 4 or 5, further characterized in that each of the primers used in the amplification contains sequences of genomic sequences that participate in gene regulation and/or transcribed and/or translated genomic sequences, as would be present after a treatment according to step a).

7. The method according to one of claims 3 to 6, further characterized in that at least one of the oligonucleotides used in step b) contains fewer nucleobases than would be necessary for a sequence-specific hybridization to the chemically treated genomic DNA sample.

8. The method according to one of claims 3 to 7, further characterized in that at least one of the oligonucleotides used in step b) of claim 3 is shorter than 18 nucleobases.

9. The method according to one of claims 3 to 7, further characterized in that at least one of the oligonucleotides used in step b) of claim 3 is shorter than 15 nucleobases.

10. The method according to one of claims 3 to 9, further characterized in that at least 4 different oligonucleotides are used simultaneously for the amplification in step b) of claim 3.

11. The method according to one of claims 3 to 9, further characterized in that more than 26 different oligonucleotides are used simultaneously for the amplification in step b) of claim 3.

12. The method according to one of claims 3 to 11, further characterized in that two oligonucleotides or two classes of oligonucleotides are used for the amplification of the DNA described in step b) of claim 3, one of which or one class of which can contain the bases C, A and T but not the base G except in the CpG context, and the other of which or the other class of which can contain the bases G, A and T but not the base C except in the CpG context.

13. The method according to one of claims 3 to 12, further characterized in that the investigation of the CpG dinucleotides contained in the amplified fragments is produced completely or partially according to claim 3d) by hybridization of the fragments, which have already been provided in the amplification with a detectable label, to an oligonucleotide array (DNA chip).

14. The method according to claim 13, further characterized in that the labels are fluorescent labels.

15. The method according to claim 13, further characterized in that the labels are radionuclides.

16. The method according to claim 13, further characterized in that the labels are detachable mass labels, which are detected in a mass spectrometer.

17. The method according to one of claims 3 to 16, further characterized in that the amplified fragments are immobilized on a surface.

18. The method according to one of claims 3 to 16, further characterized in that the hybridization is conducted with a combinatory library of distinguishable oligonucleotide or PNA oligomer probes.

19. The method according to claim 17, further characterized in that the probes are detected based on their unequivocal mass by means of matrix-assisted laser desorption/ionization mass spectrometry (MALDI-MS).

20. The method according to claim 17 or 19, further characterized in that each of the probes bears a single positive or negative net charge.

21. The method according to claims 17 to 20, further characterized in that the probes used are PNA, alkylphosphonate DNA, phosphorothioate DNA or alkylated phosphorothioate DNA.

22. The method according to one of claims 3 to 21, further characterized in that the amplification as described in step b) of claim 1 is conducted by means of a polymerase chain reaction, in which the size of the amplified fragments is limited to shortened chain extension steps of less than 30 s.

23. The method according to claims 3 to 12 and 17, further characterized in that the solid support is selected from a group comprised of beads, capillaries, planar support materials, membranes, wafers, silicon, glass, polystyrene, aluminum, steel, iron, copper, nickel, silver or gold.

24. The method according to one of claims 3 to 23, further characterized in that after the amplification according to step b) of claim 3, the products are separated by gel eletrophoresis and the fragments that are smaller than 2000 base pairs or smaller than a random limiting value below 2000 base pairs, are separated by eliminating the other products of the amplification prior to the evaluation according to step c) of claim 1.

25. The method according to claim 24, further characterized in that after the separation of amplified products of specific size, these products are amplified once more prior to conducting step c) of claim 1.

26. The method according to one of claims 3 to 25, further characterized in that methylation analyses of the upper and lower DNA strands are conducted simultaneously.

27. The method according to one of the preceding claims, further characterized in that a heat-stable DNA polymerase is selected from the following group: Taq DNA polymerase, AmpliTaq FS DNA polymerase, Deep Vent (exo.sup.-) DNA polymerase, Vent DNA polymerase, Vent (exo.sup.-) DNA polymerase and Deep Vent DNA polymerase, Thermo Sequenase, exo(-) Pseudococcus furiosus (Pfu) DNA polymerase, AmpliTaq, Ultman, 9 degree Nm, Tth, Hot Tub, Pyrococcus furiosus (Pfu) and Pyrococcus woesei (Pwo) DNA polymerase.

28. A kit, containing at least two pairs of primers, reagents and adjuvants for the amplification and/or reagents and adjuvants for the chemical treatment according to claim 3a) and/or a combinatory probe library and/or an oligonucleotide array (DNA chip) as long as they are necessary or useful for conducting the method according to the invention.


SEQUENCE LISTING (396 sequences)

SEQ ID NO: 1 - DNA, 14 bp, Artificial Sequence
  Oligonucleotide
ddddgatgttdddd

SEQ ID NO: 2 - DNA, 14 bp, Artificial Sequence
  Oligonucleotide
ddddgacgttdddd

SEQ ID NO: 3 - DNA, 14 bp, Artificial Sequence
  Oligonucleotide
hhhhaacatchhhh

SEQ ID NO: 4 - DNA, 14 bp, Artificial Sequence
  Oligonucleotide
hhhhaacgtchhhh

SEQ ID NO: 5 - DNA, 14 bp, Artificial Sequence
  Oligonucleotide
ddddttgtgadddd

SEQ ID NO: 6 - DNA, 14 bp, Artificial Sequence
  Oligonucleotide
ddddttgcgadddd

SEQ ID NO: 7 - DNA, 14 bp, Artificial Sequence
  Oligonucleotide
hhhhtcacaahhhh

SEQ ID NO: 8 - DNA, 14 bp, Artificial Sequence
  Oligonucleotide
hhhhtcgcaahhhh

SEQ ID NO: 9 - DNA, 14 bp, Artificial Sequence
  Oligonucleotide
ddddtttgaadddd

SEQ ID NO: 10 - DNA, 14 bp, Artificial Sequence
  Oligonucleotide
hhhhttcaaahhhh

SEQ ID NO: 11 - DNA, 14 bp, Artificial Sequence
  Oligonucleotide
ddddttcgaadddd

SEQ ID NO: 12 - DNA, 14 bp, Artificial Sequence
  Oligonucleotide
hhhhttcgaahhhh

SEQ ID NO: 13 - DNA, 14 bp, Artificial Sequence
  Oligonucleotide
ddddattgatdddd

SEQ ID NO: 14 - DNA, 14 bp, Artificial Sequence
  Oligonucleotide
hhhhatcaathhhh

SEQ ID NO: 15 - DNA, 14 bp, Artificial Sequence
  Oligonucleotide
ddddatcgatdddd

SEQ ID NO: 16 - DNA, 14 bp, Artificial Sequence
  Oligonucleotide
hhhhatcgathhhh

SEQ ID NO: 17 - DNA, 14 bp, Artificial Sequence
  Oligonucleotide
dddtggwttgdddd

SEQ ID NO: 18 - DNA, 14 bp, Artificial Sequence
  Oligonucleotide
ddddtggwttgddd

SEQ ID NO: 19 - DNA, 14 bp, Artificial Sequence
  Oligonucleotide
dddcggwttgdddd

SEQ ID NO: 20 - DNA, 14 bp, Artificial Sequence
  Oligonucleotide
ddddcggwttgddd

SEQ ID NO: 21 - DNA, 14 bp, Artificial Sequence
  Oligonucleotide
dddtggwtcgdddd

SEQ ID NO: 22 - DNA, 14 bp, Artificial Sequence
  Oligonucleotide
ddddtggwtcgddd

SEQ ID NO: 23 - DNA, 14 bp, Artificial Sequence
  Oligonucleotide
dddcggwtcgdddd

SEQ ID NO: 24 - DNA, 14 bp, Artificial Sequence
  Oligonucleotide
ddddcggwtcgddd

SEQ ID NO: 25 - DNA, 14 bp, Artificial Sequence
  Oligonucleotide
hhhcaasccahhhh

SEQ ID NO: 26 - DNA, 14 bp, Artificial Sequence
  Oligonucleotide
hhhhcaasccahhh

SEQ ID NO: 27 - DNA, 14 bp, Artificial Sequence
  Oligonucleotide
hhhcaasccghhhh

SEQ ID NO: 28 - DNA, 14 bp, Artificial Sequence
  Oligonucleotide
hhhhcaasccghhh

SEQ ID NO: 29 - DNA, 14 bp, Artificial Sequence
  Oligonucleotide
hhhcgasccahhhh

SEQ ID NO: 30 - DNA, 14 bp, Artificial Sequence
  Oligonucleotide
hhhhcgasccahhh

SEQ ID NO: 31 - DNA, 14 bp, Artificial Sequence
  Oligonucleotide
hhhcgasccghhhh

SEQ ID NO: 32 - DNA, 14 bp, Artificial Sequence
  Oligonucleotide
hhhhcgasccghhh

SEQ ID NO: 33 - DNA, 14 bp, Artificial Sequence
  Oligonucleotide
ddddagtgttdddd

SEQ ID NO: 34 - DNA, 14 bp, Artificial Sequence
  Oligonucleotide
ddddagcgttdddd

SEQ ID NO: 35 - DNA, 14 bp, Artificial Sequence
  Oligonucleotide
hhhhaacacthhhh

SEQ ID NO: 36 - DNA, 14 bp, Artificial Sequence
  Oligonucleotide
hhhhaacgcthhhh

SEQ ID NO: 37 - DNA, 14 bp, Artificial Sequence
  Oligonucleotide
ddddtatgtgdddd

SEQ ID NO: 38 - DNA, 14 bp, Artificial Sequence
  Oligonucleotide
ddddtacgtgdddd

SEQ ID NO: 39 - DNA, 14 bp, Artificial Sequence
  Oligonucleotide
hhhhcacatahhhh

SEQ ID NO: 40 - DNA, 14 bp, Artificial Sequence
  Oligonucleotide
hhhhcacgtahhhh

SEQ ID NO: 41 - DNA, 14 bp, Artificial Sequence
  Oligonucleotide
ddddtatgtadddd

SEQ ID NO: 42 - DNA, 14 bp, Artificial Sequence
  Oligonucleotide
hhhhtacatahhhh

SEQ ID NO: 43 - DNA, 14 bp, Artificial Sequence
  Oligonucleotide
ddddtacgtadddd

SEQ ID NO: 44 - DNA, 14 bp, Artificial Sequence
  Oligonucleotide
hhhhtacgtahhhh

SEQ ID NO: 45 - DNA, 14 bp, Artificial Sequence
  Oligonucleotide
ddddtttggadddd

SEQ ID NO: 46 - DNA, 14 bp, Artificial Sequence
  Oligonucleotide
ddddttcggadddd

SEQ ID NO: 47 - DNA, 14 bp, Artificial Sequence
  Oligonucleotide
hhhhtccaaahhhh

SEQ ID NO: 48 - DNA, 14 bp, Artificial Sequence
  Oligonucleotide
hhhhtccgaahhhh

SEQ ID NO: 49 - DNA, 14 bp, Artificial Sequence
  Oligonucleotide
ddddatgtgtdddd

SEQ ID NO: 50 - DNA, 14 bp, Artificial Sequence
  Oligonucleotide
hhhhacacathhhh

SEQ ID NO: 51 - DNA, 14 bp, Artificial Sequence
  Oligonucleotide
ddddatgtgtdddd

SEQ ID NO: 52 - DNA, 14 bp, Artificial Sequence
  Oligonucleotide
hhhhacgcgthhhh

SEQ ID NO: 53 - DNA, 14 bp, Artificial Sequence
  Oligonucleotide
dddgtggttgtddd

SEQ ID NO: 54 - DNA, 14 bp, Artificial Sequence
  Oligonucleotide
dddgcggttgtddd

SEQ ID NO: 55 - DNA, 14 bp, Artificial Sequence
  Oligonucleotide
dddgcggtcgtddd

SEQ ID NO: 56 - DNA, 14 bp, Artificial Sequence
  Oligonucleotide
dddgtggtcgtddd

SEQ ID NO: 57 - DNA, 14 bp, Artificial Sequence
  Oligonucleotide
hhhacaaccachhh

SEQ ID NO: 58 - DNA, 14 bp, Artificial Sequence
  Oligonucleotide
hhhacaaccgchhh

SEQ ID NO: 59 - DNA, 14 bp, Artificial Sequence
  Oligonucleotide
hhhacgaccgchhh

SEQ ID NO: 60 - DNA, 14 bp, Artificial Sequence
  Oligonucleotide
hhhacgaccachhh

SEQ ID NO: 61 - DNA, 14 bp, Artificial Sequence
  Oligonucleotide
ddddtgtgtadddd

SEQ ID NO: 62 - DNA, 14 bp, Artificial Sequence
  Oligonucleotide
ddddtgcgtadddd

SEQ ID NO: 63 - DNA, 14 bp, Artificial Sequence
  Oligonucleotide
hhhhtacacahhhh

SEQ ID NO: 64 - DNA, 14 bp, Artificial Sequence
  Oligonucleotide
hhhhtacgcahhhh

SEQ ID NO: 65 - DNA, 14 bp, Artificial Sequence
  Oligonucleotide
ddddgtgtgtdddd

SEQ ID NO: 66 - DNA, 14 bp, Artificial Sequence
  Oligonucleotide
hhhhacacachhhh

SEQ ID NO: 67 - DNA, 14 bp, Artificial Sequence
  Oligonucleotide
ddddgcgcgtdddd

SEQ ID NO: 68 - DNA, 14 bp, Artificial Sequence
  Oligonucleotide
hhhhacgcgchhhh

SEQ ID NO: 69 - DNA, 14 bp, Artificial Sequence
  Oligonucleotide
ddddtgtatgdddd

SEQ ID NO: 70 - DNA, 14 bp, Artificial Sequence
  Oligonucleotide
ddddcgtatgdddd

SEQ ID NO: 71 - DNA, 14 bp, Artificial Sequence
  Oligonucleotide
ddddtgtacgdddd

SEQ ID NO: 72 - DNA, 14 bp, Artificial Sequence
  Oligonucleotide
ddddcgtacgdddd

SEQ ID NO: 73 - DNA, 14 bp, Artificial Sequence
  Oligonucleotide
hhhhcatacahhhh

SEQ ID NO: 74 - DNA, 14 bp, Artificial Sequence
  Oligonucleotide
hhhhcatacghhhh

SEQ ID NO: 75 - DNA, 14 bp, Artificial Sequence
  Oligonucleotide
hhhhcgtacahhhh

SEQ ID NO: 76 - DNA, 14 bp, Artificial Sequence
  Oligonucleotide
hhhhcgtacghhhh

SEQ ID NO: 77 - DNA, 14 bp, Artificial Sequence
  Oligonucleotide
ddddaatgttdddd

SEQ ID NO: 78 - DNA, 14 bp, Artificial Sequence
  Oligonucleotide
hhhhaacatthhhh

SEQ ID NO: 79 - DNA, 14 bp, Artificial Sequence
  Oligonucleotide
ddddaacgttdddd

SEQ ID NO: 80 - DNA, 14 bp, Artificial Sequence
  Oligonucleotide
hhhhaacgtthhhh

SEQ ID NO: 81 - DNA, 14 bp, Artificial Sequence
  Oligonucleotide
ddddtgattgdddd

SEQ ID NO: 82 - DNA, 14 bp, Artificial Sequence
  Oligonucleotide
ddddtgatcgdddd

SEQ ID NO: 83 - DNA, 14 bp, Artificial Sequence
  Oligonucleotide
ddddcgattgdddd

SEQ ID NO: 84 - DNA, 14 bp, Artificial Sequence
  Oligonucleotide
ddddcgatcgdddd

SEQ ID NO: 85 - DNA, 14 bp, Artificial Sequence
  Oligonucleotide
hhhhcaatcahhhh

SEQ ID NO: 86 - DNA, 14 bp, Artificial Sequence
  Oligonucleotide
hhhhcgatcahhhh

SEQ ID NO: 87 - DNA, 14 bp, Artificial Sequence
  Oligonucleotide
hhhhcaatcghhhh

SEQ ID NO: 88 - DNA, 14 bp, Artificial Sequence
  Oligonucleotide
hhhhcgatcghhhh

SEQ ID NO: 89 - DNA, 14 bp, Artificial Sequence
  Oligonucleotide
ddddgttgatdddd

SEQ ID NO: 90 - DNA, 14 bp, Artificial Sequence
  Oligonucleotide
ddddgtcgatdddd

SEQ ID NO: 91 - DNA, 14 bp, Artificial Sequence
  Oligonucleotide
hhhhatcaachhhh

SEQ ID NO: 92 - DNA, 14 bp, Artificial Sequence
  Oligonucleotide
hhhhatcgachhhh

SEQ ID NO: 93 - DNA, 14 bp, Artificial Sequence
  Oligonucleotide
dddtggtattgddd

SEQ ID NO: 94 - DNA, 14 bp, Artificial Sequence
  Oligonucleotide
dddcggtatcgddd

SEQ ID NO: 95 - DNA, 14 bp, Artificial Sequence
  Oligonucleotide
dddtggtatcgddd

SEQ ID NO: 96 - DNA, 14 bp, Artificial Sequence
  Oligonucleotide
dddcggtattgddd

SEQ ID NO: 97 - DNA, 14 bp, Artificial Sequence
  Oligonucleotide
hhhcaataccahhh

SEQ ID NO: 98 - DNA, 14 bp, Artificial Sequence
  Oligonucleotide
hhhcgataccghhh

SEQ ID NO: 99 - DNA, 14 bp, Artificial Sequence
  Oligonucleotide
hhhcgataccahhh

SEQ ID NO: 100 - DNA, 14 bp, Artificial Sequence
  Oligonucleotide
hhhcaataccghhh

SEQ ID NO: 101 - DNA, 14 bp, Artificial Sequence
  Oligonucleotide
ddddtgttttdddd

SEQ ID NO: 102 - DNA, 14 bp, Artificial Sequence
  Oligonucleotide
ddddcgttttdddd

SEQ ID NO: 103 - DNA, 14 bp, Artificial Sequence
  Oligonucleotide
hhhhaaaacahhhh

SEQ ID NO: 104 - DNA, 14 bp, Artificial Sequence
  Oligonucleotide
hhhhaaaacghhhh

SEQ ID NO: 105 - DNA, 14 bp, Artificial Sequence
  Oligonucleotide
dddgtgtatgdddd

SEQ ID NO: 106 - DNA, 14 bp, Artificial Sequence
  Oligonucleotide
ddddgtgtatgddd

SEQ ID NO: 107 - DNA, 14 bp, Artificial Sequence
  Oligonucleotide
dddgtgtacgdddd

SEQ ID NO: 108 - DNA, 14 bp, Artificial Sequence
  Oligonucleotide
ddddgtgtacgddd

SEQ ID NO: 109 - DNA, 14 bp, Artificial Sequence
  Oligonucleotide
hhhcatacachhhh

SEQ ID NO: 110 - DNA, 14 bp, Artificial Sequence
  Oligonucleotide
hhhhcatacachhh

SEQ ID NO: 111 - DNA, 14 bp, Artificial Sequence
  Oligonucleotide
hhhcgtacachhhh

SEQ ID NO: 112 - DNA, 14 bp, Artificial Sequence
  Oligonucleotide
hhhhcgtacachhh

SEQ ID NO: 113 - DNA, 14 bp, Artificial Sequence
  Oligonucleotide
ddddgattgddddd

SEQ ID NO: 114 - DNA, 14 bp, Artificial Sequence
  Oligonucleotide
dddddgattgdddd

SEQ ID NO: 115 - DNA, 14 bp, Artificial Sequence
  Oligonucleotide
ddddgatcgddddd

SEQ ID NO: 116 - DNA, 14 bp, Artificial Sequence
  Oligonucleotide
dddddgatcgdddd

SEQ ID NO: 117 - DNA, 14 bp, Artificial Sequence
  Oligonucleotide
hhhhcaatchhhhh

SEQ ID NO: 118 - DNA, 14 bp, Artificial Sequence
  Oligonucleotide
hhhhhcaatchhhh

SEQ ID NO: 119 - DNA, 14 bp, Artificial Sequence
  Oligonucleotide
hhhhctagchhhhh

SEQ ID NO: 120 - DNA, 14 bp, Artificial Sequence
  Oligonucleotide
hhhhhctagchhhh

SEQ ID NO: 121 - DNA, 14 bp, Artificial Sequence
  Oligonucleotide
dddtgtatatgddd

SEQ ID NO: 122 - DNA, 14 bp, Artificial Sequence
  Oligonucleotide
dddtgtatacgddd

SEQ ID NO: 123 - DNA, 14 bp, Artificial Sequence
  Oligonucleotide
dddcgtatatgddd

SEQ ID NO: 124 - DNA, 14 bp, Artificial Sequence
  Oligonucleotide
dddcgtatacgddd

SEQ ID NO: 125 - DNA, 14 bp, Artificial Sequence
  Oligonucleotide
hhhcatatacahhh

SEQ ID NO: 126 - DNA, 14 bp, Artificial Sequence
  Oligonucleotide
hhhcgtatacahhh

SEQ ID NO: 127 - DNA, 14 bp, Artificial Sequence
  Oligonucleotide
hhhcatatacghhh

SEQ ID NO: 128 - DNA, 14 bp, Artificial Sequence
  Oligonucleotide
hhhcgtatacghhh

SEQ ID NO: 129 - DNA, 14 bp, Artificial Sequence
  Oligonucleotide
dddtgagtttgddd

SEQ ID NO: 130 - DNA, 14 bp, Artificial Sequence
  Oligonucleotide
dddtgagttcgddd

SEQ ID NO: 131 - DNA, 14 bp, Artificial Sequence
  Oligonucleotide
dddcgagtttgddd

SEQ ID NO: 132 - DNA, 14 bp, Artificial Sequence
  Oligonucleotide
dddcgagttcgddd

SEQ ID NO: 133 - DNA, 14 bp, Artificial Sequence
  Oligonucleotide
hhhcaaactcahhh

SEQ ID NO: 134 - DNA, 14 bp, Artificial Sequence
  Oligonucleotide
hhhcgaactcahhh

SEQ ID NO: 135 - DNA, 14 bp, Artificial Sequence
  Oligonucleotide
hhhcaaactcghhh

SEQ ID NO: 136 - DNA, 14 bp, Artificial Sequence
  Oligonucleotide
hhhcgaactcghhh

SEQ ID NO: 137 - DNA, 14 bp, Artificial Sequence
  Oligonucleotide
dddtgttaatgddd

SEQ ID NO: 138 - DNA, 14 bp, Artificial Sequence
  Oligonucleotide
dddcgttaatgddd

SEQ ID NO: 139 - DNA, 14 bp, Artificial Sequence
  Oligonucleotide
dddtgttaacgddd

SEQ ID NO: 140 - DNA, 14 bp, Artificial Sequence
  Oligonucleotide
dddcgttaacgddd

SEQ ID NO: 141 - DNA, 14 bp, Artificial Sequence
  Oligonucleotide
hhhcattaacahhh

SEQ ID NO: 142 - DNA, 14 bp, Artificial Sequence
  Oligonucleotide
hhhcattaacghhh

SEQ ID NO: 143 - DNA, 14 bp, Artificial Sequence
  Oligonucleotide
hhhcgttaacahhh

SEQ ID NO: 144 - DNA, 14 bp, Artificial Sequence
  Oligonucleotide
hhhcgttaacghhh

SEQ ID NO: 145 - DNA, 14 bp, Artificial Sequence
  Oligonucleotide
ddddtgtatgdddd

SEQ ID NO: 146 - DNA, 14 bp, Artificial Sequence
  Oligonucleotide
ddddtgtacgdddd

SEQ ID NO: 147 - DNA, 14 bp, Artificial Sequence
  Oligonucleotide
ddddcgtatgdddd

SEQ ID NO: 148 - DNA, 14 bp, Artificial Sequence
  Oligonucleotide
ddddcgtacgdddd

SEQ ID NO: 149 - DNA, 14 bp, Artificial Sequence
  Oligonucleotide
hhhhcatacahhhh

SEQ ID NO: 150 - DNA, 14 bp, Artificial Sequence
  Oligonucleotide
hhhhcgtacahhhh

SEQ ID NO: 151 - DNA, 14 bp, Artificial Sequence
  Oligonucleotide
hhhhcatacghhhh

SEQ ID NO: 152 - DNA, 14 bp, Artificial Sequence
  Oligonucleotide
hhhhcgtacghhhh

SEQ ID NO: 153 - DNA, 14 bp, Artificial Sequence
  Oligonucleotide
dddggtcggttddd

SEQ ID NO: 154 - DNA, 14 bp, Artificial Sequence
  Oligonucleotide
dddggttggttddd

SEQ ID NO: 155 - DNA, 14 bp, Artificial Sequence
  Oligonucleotide
hhhaaccgacchhh

SEQ ID NO: 156 - DNA, 14 bp, Artificial Sequence
  Oligonucleotide
hhhaaccaacchhh

SEQ ID NO: 157 - DNA, 14 bp, Artificial Sequence
  Oligonucleotide
ddddgacgtddddd

SEQ ID NO: 158 - DNA, 14 bp, Artificial Sequence
  Oligonucleotide
dddddgacgtdddd

SEQ ID NO: 159 - DNA, 14 bp, Artificial Sequence
  Oligonucleotide
ddddgatgtddddd

SEQ ID NO: 160 - DNA, 14 bp, Artificial Sequence
  Oligonucleotide
dddddgatgtdddd

SEQ ID NO: 161 - DNA, 14 bp, Artificial Sequence
  Oligonucleotide
hhhhacgtchhhhh

SEQ ID NO: 162 - DNA, 14 bp, Artificial Sequence
  Oligonucleotide
hhhhhacgtchhhh

SEQ ID NO: 163 - DNA, 14 bp, Artificial Sequence
  Oligonucleotide
hhhhacatchhhhh

SEQ ID NO: 164 - DNA, 14 bp, Artificial Sequence
  Oligonucleotide
hhhhhacatchhhh

SEQ ID NO: 165 - DNA, 14 bp, Artificial Sequence
  Oligonucleotide
ddddggcgttdddd

SEQ ID NO: 166 - DNA, 14 bp, Artificial Sequence
  Oligonucleotide
ddddggtgttdddd

SEQ ID NO: 167 - DNA, 14 bp, Artificial Sequence
  Oligonucleotide
hhhhaacgcchhhh

SEQ ID NO: 168 - DNA, 14 bp, Artificial Sequence
  Oligonucleotide
hhhhaacacchhhh

SEQ ID NO: 169 - DNA, 14 bp, Artificial Sequence
  Oligonucleotide
ddddgtcggtdddd

SEQ ID NO: 170 - DNA, 14 bp, Artificial Sequence
  Oligonucleotide
ddddgttggtdddd

SEQ ID NO: 171 - DNA, 14 bp, Artificial Sequence
  Oligonucleotide
hhhhaccgachhhh

SEQ ID NO: 172 - DNA, 14 bp, Artificial Sequence
  Oligonucleotide
hhhhaccaachhhh

SEQ ID NO: 173 - DNA, 13 bp, Artificial Sequence
  Oligonucleotide
ddddtgcggdddd

SEQ ID NO: 174 - DNA, 14 bp, Artificial Sequence
  Oligonucleotide
ddddttgtggdddd

SEQ ID NO: 175 - DNA, 14 bp, Artificial Sequence
  Oligonucleotide
hhhhccgcgahhhh

SEQ ID NO: 176 - DNA, 14 bp, Artificial Sequence
  Oligonucleotide
hhhhccacaahhhh

SEQ ID NO: 177 - DNA, 14 bp, Artificial Sequence
  Oligonucleotide
ddddttcgggdddd

SEQ ID NO: 178 - DNA, 14 bp, Artificial Sequence
  Oligonucleotide
ddddtttgggdddd

SEQ ID NO: 179 - DNA, 14 bp, Artificial Sequence
  Oligonucleotide
hhhhcccgaahhhh

SEQ ID NO: 180 - DNA, 14 bp, Artificial Sequence
  Oligonucleotide
hhhhcccaaahhhh

SEQ ID NO: 181 - DNA, 14 bp, Artificial Sequence
  Oligonucleotide
ddddttcgagdddd

SEQ ID NO: 182 - DNA, 14 bp, Artificial Sequence
  Oligonucleotide
ddddtttgagdddd

SEQ ID NO: 183 - DNA, 14 bp, Artificial Sequence
  Oligonucleotide
hhhhctcgaahhhh

SEQ ID NO: 184 - DNA, 14 bp, Artificial Sequence
  Oligonucleotide
hhhhctcaaahhhh

SEQ ID NO: 185 - DNA, 14 bp, Artificial Sequence
  Oligonucleotide
ddddcggtcgdddd

SEQ ID NO: 186 - DNA, 14 bp, Artificial Sequence
  Oligonucleotide
ddddtggttgdddd

SEQ ID NO: 187 - DNA, 14 bp, Artificial Sequence
  Oligonucleotide
ddddcggttgdddd

SEQ ID NO: 188 - DNA, 14 bp, Artificial Sequence
  Oligonucleotide
ddddtggtcgdddd

SEQ ID NO: 189 - DNA, 14 bp, Artificial Sequence
  Oligonucleotide
hhhhcgaccghhhh

SEQ ID NO: 190 - DNA, 14 bp, Artificial Sequence
  Oligonucleotide
hhhhcaaccahhhh

SEQ ID NO: 191 - DNA, 14 bp, Artificial Sequence
  Oligonucleotide
hhhhcaaccghhhh

SEQ ID NO: 192 - DNA, 14 bp, Artificial Sequence
  Oligonucleotide
hhhhcgaccahhhh

SEQ ID NO: 193 - DNA, 10 bp, Artificial Sequence
  Oligonucleotide
ddgatgttdd

SEQ ID NO: 194 - DNA, 10 bp, Artificial Sequence
  Oligonucleotide
ddgacgttdd

SEQ ID NO: 195 - DNA, 10 bp, Artificial Sequence
  Oligonucleotide
hhaacatchh

SEQ ID NO: 196 - DNA, 10 bp, Artificial Sequence
  Oligonucleotide
hhaacgtchh

SEQ ID NO: 197 - DNA, 10 bp, Artificial Sequence
  Oligonucleotide
ddttgtgadd

SEQ ID NO: 198 - DNA, 10 bp, Artificial Sequence
  Oligonucleotide
ddttgcgadd

SEQ ID NO: 199 - DNA, 10 bp, Artificial Sequence
  Oligonucleotide
hhtcacaahh

SEQ ID NO: 200 - DNA, 10 bp, Artificial Sequence
  Oligonucleotide
hhtcgcaahh

SEQ ID NO: 201 - DNA, 10 bp, Artificial Sequence
  Oligonucleotide
ddtttgaadd

SEQ ID NO: 202 - DNA, 10 bp, Artificial Sequence
  Oligonucleotide
hhttcaaahh

SEQ ID NO: 203 - DNA, 10 bp, Artificial Sequence
  Oligonucleotide
ddttcgaadd

SEQ ID NO: 204 - DNA, 10 bp, Artificial Sequence
  Oligonucleotide
hhttcgaahh

SEQ ID NO: 205 - DNA, 10 bp, Artificial Sequence
  Oligonucleotide
ddattgatdd

SEQ ID NO: 206 - DNA, 10 bp, Artificial Sequence
  Oligonucleotide
hhatcaathh

SEQ ID NO: 207 - DNA, 10 bp, Artificial Sequence
  Oligonucleotide
ddatcgatdd

SEQ ID NO: 208 - DNA, 10 bp, Artificial Sequence
  Oligonucleotide
hhatcgathh

SEQ ID NO: 209 - DNA, 10 bp, Artificial Sequence
  Oligonucleotide
dtggwttgdd

SEQ ID NO: 210 - DNA, 10 bp, Artificial Sequence
  Oligonucleotide
ddtggwttgd

SEQ ID NO: 211 - DNA, 10 bp, Artificial Sequence
  Oligonucleotide
dcggwttgdd

SEQ ID NO: 212 - DNA, 10 bp, Artificial Sequence
  Oligonucleotide
ddcggwttgd

SEQ ID NO: 213 - DNA, 10 bp, Artificial Sequence
  Oligonucleotide
dtggwtcgdd

SEQ ID NO: 214 - DNA, 10 bp, Artificial Sequence
  Oligonucleotide
ddtggwtcgd

SEQ ID NO: 215 - DNA, 10 bp, Artificial Sequence
  Oligonucleotide
dcggwtcgdd

SEQ ID NO: 216 - DNA, 10 bp, Artificial Sequence
  Oligonucleotide
ddcggwtcgd

SEQ ID NO: 217 - DNA, 10 bp, Artificial Sequence
  Oligonucleotide
hcaasccahh

SEQ ID NO: 218 - DNA, 10 bp, Artificial Sequence
  Oligonucleotide
hhcaasccah

SEQ ID NO: 219 - DNA, 10 bp, Artificial Sequence
  Oligonucleotide
hcaasccghh

SEQ ID NO: 220 - DNA, 10 bp, Artificial Sequence
  Oligonucleotide
hhcaasccgh

SEQ ID NO: 221 - DNA, 10 bp, Artificial Sequence
  Oligonucleotide
hcgasccahh

SEQ ID NO: 222 - DNA, 10 bp, Artificial Sequence
  Oligonucleotide
hhcgasccah

SEQ ID NO: 223 - DNA, 10 bp, Artificial Sequence
  Oligonucleotide
hcgasccghh

SEQ ID NO: 224 - DNA, 10 bp, Artificial Sequence
  Oligonucleotide
hhcgasccgh

SEQ ID NO: 225 - DNA, 10 bp, Artificial Sequence
  Oligonucleotide
ddagtgttdd

SEQ ID NO: 226 - DNA, 10 bp, Artificial Sequence
  Oligonucleotide
ddagcgttdd

SEQ ID NO: 227 - DNA, 10 bp, Artificial Sequence
  Oligonucleotide
hhaacacthh

SEQ ID NO: 228 - DNA, 10 bp, Artificial Sequence
  Oligonucleotide
hhaacgcthh

SEQ ID NO: 229 - DNA, 10 bp, Artificial Sequence
  Oligonucleotide
ddtatgtgdd

SEQ ID NO: 230 - DNA, 10 bp, Artificial Sequence
  Oligonucleotide
ddtacgtgdd

SEQ ID NO: 231 - DNA, 10 bp, Artificial Sequence
  Oligonucleotide
hhcacatahh

SEQ ID NO: 232 - DNA, 10 bp, Artificial Sequence
  Oligonucleotide
hhcacgtahh

SEQ ID NO: 233 - DNA, 10 bp, Artificial Sequence
  Oligonucleotide
ddtatgtadd

SEQ ID NO: 234 - DNA, 10 bp, Artificial Sequence
  Oligonucleotide
hhtacatahh

SEQ ID NO: 235 - DNA, 10 bp, Artificial Sequence
  Oligonucleotide
ddtacgtadd

SEQ ID NO: 236 - DNA, 10 bp, Artificial Sequence
  Oligonucleotide
hhtacgtahh

SEQ ID NO: 237 - DNA, 10 bp, Artificial Sequence
  Oligonucleotide
ddtttggadd

SEQ ID NO: 238 - DNA, 10 bp, Artificial Sequence
  Oligonucleotide
ddttcggadd

SEQ ID NO: 239 - DNA, 10 bp, Artificial Sequence
  Oligonucleotide
hhtccaaahh

SEQ ID NO: 240 - DNA, 10 bp, Artificial Sequence
  Oligonucleotide
hhtccgaahh

SEQ ID NO: 241 - DNA, 10 bp, Artificial Sequence
  Oligonucleotide
ddatgtgtdd

SEQ ID NO: 242 - DNA, 10 bp, Artificial Sequence
  Oligonucleotide
hhacacathh

SEQ ID NO: 243 - DNA, 10 bp, Artificial Sequence
  Oligonucleotide
ddatgtgtdd

SEQ ID NO: 244 - DNA, 10 bp, Artificial Sequence
  Oligonucleotide
hhacgcgthh

SEQ ID NO: 245 - DNA, 10 bp, Artificial Sequence
  Oligonucleotide
dgtggttgtd

SEQ ID NO: 246 - DNA, 10 bp, Artificial Sequence
  Oligonucleotide
dgcggttgtd

SEQ ID NO: 247 - DNA, 10 bp, Artificial Sequence
  Oligonucleotide
dgcggtcgtd

SEQ ID NO: 248 - DNA, 10 bp, Artificial Sequence
  Oligonucleotide
dgtggtcgtd

SEQ ID NO: 249 - DNA, 10 bp, Artificial Sequence
  Oligonucleotide
hacaaccach

SEQ ID NO: 250 - DNA, 10 bp, Artificial Sequence
  Oligonucleotide
hacaaccgch

SEQ ID NO: 251 - DNA, 10 bp, Artificial Sequence
  Oligonucleotide
hacgaccgch

SEQ ID NO: 252 - DNA, 10 bp, Artificial Sequence
  Oligonucleotide
hacgaccach

SEQ ID NO: 253 - DNA, 10 bp, Artificial Sequence
  Oligonucleotide
ddtgtgtadd

SEQ ID NO: 254 - DNA, 10 bp, Artificial Sequence
  Oligonucleotide
ddtgcgtadd

SEQ ID NO: 255 - DNA, 10 bp, Artificial Sequence
  Oligonucleotide
hhtacacahh

SEQ ID NO: 256 - DNA, 10 bp, Artificial Sequence
  Oligonucleotide
hhtacgcahh

SEQ ID NO: 257 - DNA, 10 bp, Artificial Sequence
  Oligonucleotide
ddgtgtgtdd

SEQ ID NO: 258 - DNA, 10 bp, Artificial Sequence
  Oligonucleotide
hhacacachh

SEQ ID NO: 259 - DNA, 10 bp, Artificial Sequence
  Oligonucleotide
ddgcgcgtdd

SEQ ID NO: 260 - DNA, 10 bp, Artificial Sequence
  Oligonucleotide
hhacgcgchh

SEQ ID NO: 261 - DNA, 10 bp, Artificial Sequence
  Oligonucleotide
ddtgtatgdd

SEQ ID NO: 262 - DNA, 10 bp, Artificial Sequence
  Oligonucleotide
ddcgtatgdd

SEQ ID NO: 263 - DNA, 10 bp, Artificial Sequence
  Oligonucleotide
ddtgtacgdd

SEQ ID NO: 264 - DNA, 10 bp, Artificial Sequence
  Oligonucleotide
ddcgtacgdd

SEQ ID NO: 265 - DNA, 10 bp, Artificial Sequence
  Oligonucleotide
hhcatacahh

SEQ ID NO: 266 - DNA, 10 bp, Artificial Sequence
  Oligonucleotide
hhcatacghh

SEQ ID NO: 267 - DNA, 10 bp, Artificial Sequence
  Oligonucleotide
hhcgtacahh

SEQ ID NO: 268 - DNA, 10 bp, Artificial Sequence
  Oligonucleotide
hhcgtacghh

SEQ ID NO: 269 - DNA, 10 bp, Artificial Sequence
  Oligonucleotide
ddaatgttdd

SEQ ID NO: 270 - DNA, 10 bp, Artificial Sequence
  Oligonucleotide
hhaacatthh

SEQ ID NO: 271 - DNA, 10 bp, Artificial Sequence
  Oligonucleotide
ddaacgttdd

SEQ ID NO: 272 - DNA, 10 bp, Artificial Sequence
  Oligonucleotide
hhaacgtthh

SEQ ID NO: 273 - DNA, 10 bp, Artificial Sequence
  Oligonucleotide
ddtgattgdd

SEQ ID NO: 274 - DNA, 10 bp, Artificial Sequence
  Oligonucleotide
ddtgatcgdd

SEQ ID NO: 275 - DNA, 10 bp, Artificial Sequence
  Oligonucleotide
ddcgattgdd

SEQ ID NO: 276 - DNA, 10 bp, Artificial Sequence
  Oligonucleotide
ddcgatcgdd

SEQ ID NO: 277 - DNA, 10 bp, Artificial Sequence
  Oligonucleotide
hhcaatcahh

SEQ ID NO: 278 - DNA, 10 bp, Artificial Sequence
  Oligonucleotide
hhcgatcahh

SEQ ID NO: 279 - DNA, 10 bp, Artificial Sequence
  Oligonucleotide
hhcaatcghh

SEQ ID NO: 280 - DNA, 10 bp, Artificial Sequence
  Oligonucleotide
hhcgatcghh

SEQ ID NO: 281 - DNA, 10 bp, Artificial Sequence
  Oligonucleotide
ddgttgatdd

SEQ ID NO: 282 - DNA, 10 bp, Artificial Sequence
  Oligonucleotide
ddgtcgatdd

SEQ ID NO: 283 - DNA, 10 bp, Artificial Sequence
  Oligonucleotide
hhatcaachh

SEQ ID NO: 284 - DNA, 10 bp, Artificial Sequence
  Oligonucleotide
hhatcgachh

SEQ ID NO: 285 - DNA, 10 bp, Artificial Sequence
  Oligonucleotide
dtggtattgd

SEQ ID NO: 286 - DNA, 10 bp, Artificial Sequence
  Oligonucleotide
dcggtatcgd

SEQ ID NO: 287 - DNA, 10 bp, Artificial Sequence
  Oligonucleotide
dtggtatcgd

SEQ ID NO: 288 - DNA, 10 bp, Artificial Sequence
  Oligonucleotide
dcggtattgd

SEQ ID NO: 289 - DNA, 10 bp, Artificial Sequence
  Oligonucleotide
hcaataccah

SEQ ID NO: 290 - DNA, 10 bp, Artificial Sequence
  Oligonucleotide
hcgataccgh

SEQ ID NO: 291 - DNA, 10 bp, Artificial Sequence
  Oligonucleotide
hcgataccah

SEQ ID NO: 292 - DNA, 10 bp, Artificial Sequence
  Oligonucleotide
hcaataccgh

SEQ ID NO: 293 - DNA, 10 bp, Artificial Sequence
  Oligonucleotide
ddtgttttdd

SEQ ID NO: 294 - DNA, 10 bp, Artificial Sequence
  Oligonucleotide
ddcgttttdd

SEQ ID NO: 295 - DNA, 10 bp, Artificial Sequence
  Oligonucleotide
hhaaaacahh

SEQ ID NO: 296 - DNA, 10 bp, Artificial Sequence
  Oligonucleotide
hhaaaacghh

SEQ ID NO: 297 - DNA, 10 bp, Artificial Sequence
  Oligonucleotide
dgtgtatgdd

SEQ ID NO: 298 - DNA, 10 bp, Artificial Sequence
  Oligonucleotide
ddgtgtatgd

SEQ ID NO: 299 - DNA, 10 bp, Artificial Sequence
  Oligonucleotide
dgtgtacgdd

SEQ ID NO: 300 - DNA, 10 bp, Artificial Sequence
  Oligonucleotide
ddgtgtacgd

SEQ ID NO: 301 - DNA, 12 bp, Artificial Sequence
  Oligonucleotide
hhhcatacachh

SEQ ID NO: 302 - DNA, 10 bp, Artificial Sequence
  Oligonucleotide
hhcatacach

SEQ ID NO: 303 - DNA, 10 bp, Artificial Sequence
  Oligonucleotide
hcgtacachh

SEQ ID NO: 304 - DNA, 10 bp, Artificial Sequence
  Oligonucleotide
hhcgtacach

SEQ ID NO: 305 - DNA, 10 bp, Artificial Sequence
  Oligonucleotide
ddgattgddd

SEQ ID NO: 306 - DNA, 10 bp, Artificial Sequence
  Oligonucleotide
dddgattgdd

SEQ ID NO: 307 - DNA, 10 bp, Artificial Sequence
  Oligonucleotide
ddgatcgddd

SEQ ID NO: 308 - DNA, 10 bp, Artificial Sequence
  Oligonucleotide
dddgatcgdd

SEQ ID NO: 309 - DNA, 10 bp, Artificial Sequence
  Oligonucleotide
hhcaatchhh

SEQ ID NO: 310 - DNA, 10 bp, Artificial Sequence
  Oligonucleotide
hhhcaatchh

SEQ ID NO: 311 - DNA, 10 bp, Artificial Sequence
  Oligonucleotide
hhctagchhh

SEQ ID NO: 312 - DNA, 10 bp, Artificial Sequence
  Oligonucleotide
hhhctagchh

SEQ ID NO: 313 - DNA, 10 bp, Artificial Sequence
  Oligonucleotide
dtgtatatgd

SEQ ID NO: 314 - DNA, 10 bp, Artificial Sequence
  Oligonucleotide
dtgtatacgd

SEQ ID NO: 315 - DNA, 10 bp, Artificial Sequence
  Oligonucleotide
dcgtatatgd

SEQ ID NO: 316 - DNA, 10 bp, Artificial Sequence
  Oligonucleotide
dcgtatacgd

SEQ ID NO: 317 - DNA, 10 bp, Artificial Sequence
  Oligonucleotide
hcatatacah

SEQ ID NO: 318 - DNA, 10 bp, Artificial Sequence
  Oligonucleotide
hcgtatacah

SEQ ID NO: 319 - DNA, 10 bp, Artificial Sequence
  Oligonucleotide
hcatatacgh

SEQ ID NO: 320 - DNA, 10 bp, Artificial Sequence
  Oligonucleotide
hcgtatacgh

SEQ ID NO: 321 - DNA, 10 bp, Artificial Sequence
  Oligonucleotide
dtgagtttgd

SEQ ID NO: 322 - DNA, 10 bp, Artificial Sequence
  Oligonucleotide
dtgagttcgd

SEQ ID NO: 323 - DNA, 10 bp, Artificial Sequence
  Oligonucleotide
dcgagtttgd

SEQ ID NO: 324 - DNA, 10 bp, Artificial Sequence
  Oligonucleotide
dcgagttcgd

SEQ ID NO: 325 - DNA, 10 bp, Artificial Sequence
  Oligonucleotide
hcaaactcah

SEQ ID NO: 326 - DNA, 10 bp, Artificial Sequence
  Oligonucleotide
hcgaactcah

SEQ ID NO: 327 - DNA, 10 bp, Artificial Sequence
  Oligonucleotide
hcaaactcgh

SEQ ID NO: 328 - DNA, 10 bp, Artificial Sequence
  Oligonucleotide
hcgaactcgh

SEQ ID NO: 329 - DNA, 10 bp, Artificial Sequence
  Oligonucleotide
dtgttaatgd

SEQ ID NO: 330 - DNA, 10 bp, Artificial Sequence
  Oligonucleotide
dcgttaatgd

SEQ ID NO: 331 - DNA, 10 bp, Artificial Sequence
  Oligonucleotide
dtgttaacgd

SEQ ID NO: 332 - DNA, 10 bp, Artificial Sequence
  Oligonucleotide
dcgttaacgd

SEQ ID NO: 333 - DNA, 10 bp, Artificial Sequence
  Oligonucleotide
hcattaacah

SEQ ID NO: 334 - DNA, 10 bp, Artificial Sequence
  Oligonucleotide
hcattaacgh

SEQ ID NO: 335 - DNA, 10 bp, Artificial Sequence
  Oligonucleotide
hcgttaacah

SEQ ID NO: 336 - DNA, 10 bp, Artificial Sequence
  Oligonucleotide
hcgttaacgh

SEQ ID NO: 337 - DNA, 10 bp, Artificial Sequence
  Oligonucleotide
ddtgtatgdd

SEQ ID NO: 338 - DNA, 10 bp, Artificial Sequence
  Oligonucleotide
ddtgtacgdd

SEQ ID NO: 339 - DNA, 10 bp, Artificial Sequence
  Oligonucleotide
ddcgtatgdd

SEQ ID NO: 340 - DNA, 10 bp, Artificial Sequence
  Oligonucleotide
ddcgtacgdd

SEQ ID NO: 341 - DNA, 10 bp, Artificial Sequence
  Oligonucleotide
hhcatacahh

SEQ ID NO: 342 - DNA, 10 bp, Artificial Sequence
  Oligonucleotide
hhcgtacahh

SEQ ID NO: 343 - DNA, 10 bp, Artificial Sequence
  Oligonucleotide
hhcatacghh

SEQ ID NO: 344 - DNA, 10 bp, Artificial Sequence
  Oligonucleotide
hhcgtacghh

SEQ ID NO: 345 - DNA, 10 bp, Artificial Sequence
  Oligonucleotide
dggtcggttd

SEQ ID NO: 346 - DNA, 10 bp, Artificial Sequence
  Oligonucleotide
dggttggttd

SEQ ID NO: 347 - DNA, 10 bp, Artificial Sequence
  Oligonucleotide
haaccgacch

SEQ ID NO: 348 - DNA, 10 bp, Artificial Sequence
  Oligonucleotide
haaccaacch

SEQ ID NO: 349 - DNA, 10 bp, Artificial Sequence
  Oligonucleotide
ddgacgtddd

SEQ ID NO: 350 - DNA, 10 bp, Artificial Sequence
  Oligonucleotide
dddgacgtdd

SEQ ID NO: 351 - DNA, 10 bp, Artificial Sequence
  Oligonucleotide
ddgatgtddd

SEQ ID NO: 352 - DNA, 10 bp, Artificial Sequence
  Oligonucleotide
dddgatgtdd

SEQ ID NO: 353 - DNA, 10 bp, Artificial Sequence
  Oligonucleotide
hhacgtchhh

SEQ ID NO: 354 - DNA, 10 bp, Artificial Sequence
  Oligonucleotide
hhhacgtchh

SEQ ID NO: 355 - DNA, 10 bp, Artificial Sequence
  Oligonucleotide
hhacatchhh

SEQ ID NO: 356 - DNA, 10 bp, Artificial Sequence
  Oligonucleotide
hhhacatchh

SEQ ID NO: 357 - DNA, 10 bp, Artificial Sequence
  Oligonucleotide
ddggcgttdd

SEQ ID NO: 358 - DNA, 10 bp, Artificial Sequence
  Oligonucleotide
ddggtgttdd

SEQ ID NO: 359 - DNA, 10 bp, Artificial Sequence
  Oligonucleotide
hhaacgcchh

SEQ ID NO: 360 - DNA, 10 bp, Artificial Sequence
  Oligonucleotide
hhaacacchh

SEQ ID NO: 361 - DNA, 10 bp, Artificial Sequence
  Oligonucleotide
ddgtcggtdd

SEQ ID NO: 362 - DNA, 10 bp, Artificial Sequence
  Oligonucleotide
ddgttggtdd

SEQ ID NO: 363 - DNA, 10 bp, Artificial Sequence
  Oligonucleotide
hhaccgachh

SEQ ID NO: 364 - DNA, 10 bp, Artificial Sequence
  Oligonucleotide
hhaccaachh

SEQ ID NO: 365 - DNA, 9 bp, Artificial Sequence
  Oligonucleotide
ddtgcggdd

SEQ ID NO: 366 - DNA, 10 bp, Artificial Sequence
  Oligonucleotide
ddttgtggdd

SEQ ID NO: 367 - DNA, 10 bp, Artificial Sequence
  Oligonucleotide
hhccgcgahh

SEQ ID NO: 368 - DNA, 10 bp, Artificial Sequence
  Oligonucleotide
hhccacaahh

SEQ ID NO: 369 - DNA, 10 bp, Artificial Sequence
  Oligonucleotide
ddttcgggdd

SEQ ID NO: 370 - DNA, 10 bp, Artificial Sequence
  Oligonucleotide
ddtttgggdd

SEQ ID NO: 371 - DNA, 10 bp, Artificial Sequence
  Oligonucleotide
hhcccgaahh

SEQ ID NO: 372 - DNA, 10 bp, Artificial Sequence
  Oligonucleotide
hhcccaaahh

SEQ ID NO: 373 - DNA, 10 bp, Artificial Sequence
  Oligonucleotide
ddttcgagdd

SEQ ID NO: 374 - DNA, 10 bp, Artificial Sequence
  Oligonucleotide
ddtttgagdd

SEQ ID NO: 375 - DNA, 10 bp, Artificial Sequence
  Oligonucleotide
hhctcgaahh

SEQ ID NO: 376 - DNA, 10 bp, Artificial Sequence
  Oligonucleotide
hhctcaaahh

SEQ ID NO: 377 - DNA, 10 bp, Artificial Sequence
  Oligonucleotide
ddcggtcgdd

SEQ ID NO: 378 - DNA, 10 bp, Artificial Sequence
  Oligonucleotide
ddtggttgdd

SEQ ID NO: 379 - DNA, 10 bp, Artificial Sequence
  Oligonucleotide
ddcggttgdd

SEQ ID NO: 380 - DNA, 10 bp, Artificial Sequence
  Oligonucleotide
ddtggtcgdd

SEQ ID NO: 381 - DNA, 10 bp, Artificial Sequence
  Oligonucleotide
hhcgaccghh

SEQ ID NO: 382 - DNA, 10 bp, Artificial Sequence
  Oligonucleotide
hhcaaccahh

SEQ ID NO: 383 - DNA, 10 bp, Artificial Sequence
  Oligonucleotide
hhcaaccghh

SEQ ID NO: 384 - DNA, 10 bp, Artificial Sequence
  Oligonucleotide
hhcgaccahh

SEQ ID NO: 385 - DNA, 20 bp, Artificial Sequence
  Oligonucleotide
gtttagaagtttaagattag

SEQ ID NO: 386 - DNA, 20 bp, Artificial Sequence
  Oligonucleotide
caaaaactcaacctctatct

SEQ ID NO: 387 - DNA, 18 bp, Artificial Sequence
  Oligonucleotide
tttttagtcgattttaga

SEQ ID NO: 388 - DNA, 18 bp, Artificial Sequence
  Oligonucleotide
tttttagttgattttaga

SEQ ID NO: 389 - DNA, 20 bp, Artificial Sequence
  Oligonucleotide
atttggagttgaagtatttg

SEQ ID NO: 390 - DNA, 20 bp, Artificial Sequence
  Oligonucleotide
aactatacccaaacacctac

SEQ ID NO: 391 - DNA, 18 bp, Artificial Sequence
  Oligonucleotide
tgtttatgcgtatttgtt

SEQ ID NO: 392 - DNA, 18 bp, Artificial Sequence
  Oligonucleotide
tgtttatgtgtatttgtt

SEQ ID NO: 393 - DNA, 28 bp, Artificial Sequence
  Oligonucleotide
agggagttttttttagggaatagaggga

SEQ ID NO: 394 - DNA, 28 bp, Artificial Sequence
  Oligonucleotide
taatcccaaaacctctccactacaacaa

SEQ ID NO: 395 - DNA, 11 bp, Artificial Sequence
  Oligonucleotide
caaacgttcaa

SEQ ID NO: 396 - DNA, 11 bp, Artificial Sequence
  Oligonucleotide
caaacattcaa